(19) United States
(12) Patent Application Publication
(10) Pub. No.: US 2021/0379203 A1
(43) Pub. Date: Dec. 9, 2021

(54) New Tools for Improving Gene Therapy and Use Thereof

(71) Applicant: Vrije Universiteit Brussel, Brussel (BE)

(72) Inventors: Thierry Vandendriessche, Bierbeek (BE); Lay Khim Chuah, Bierbeek (BE)

(21) Appl. No.: 17/288,442

(22) PCT Filed: Oct. 28, 2019
(86) PCT No.: PCT/EP2019/079351

§ 371 Date: Apr. 23, 2021

(30) Foreign Application Priority Data
Oct. 26, 2018 (EP) 18202888.6

(51) Int. Cl. A61K 48/00; C07K 14/76; C12N 9/64; C07K 14/755; C12N 15/86; A61P 1/16

(52) U.S. Cl. CPC A61K 48/0058; C07K 14/76; C12N 9/644; C12Y 304/21022; C07K 14/755; C07K 2319/31; A61P 1/16; C12N 2750/14143; C12N 2800/22; C12N 2830/008; C12N 2830/42; C12N 15/86

(57) ABSTRACT

The present invention relates to a nucleic acid molecule encoding human albumin for increasing the levels and/or activity of a protein or polypeptide encoded by a transgene, comprising a sequence defined by SEQ ID NO: 14 or a sequence having at least 80% sequence identity to said sequence, its use in nucleic acid expression cassettes and vectors containing liver-specific regulatory elements and codon-optimized factor IX, factor VIII, factor VII or factor VIIa transgenes, methods employing these expression cassettes and vectors and uses thereof. The present invention is particularly useful for applications using liver-directed gene therapy, in particular for the treatment of hemophilia A, hemophilia B or factor VII deficiency.

### FIELD OF THE INVENTION

The present invention relates to new tools that are able to enhance tissue-specific expression and/or activity of (trans)genes, methods employing these tools and uses thereof. The invention further encompasses expression systems and pharmaceutical compositions comprising these. The present invention is particularly useful for applications using gene therapy, more particularly tissue-directed gene therapy, and for vaccination purposes.

### BACKGROUND OF THE INVENTION

Hemophilia B is an X-linked, recessive bleeding disorder caused by deficiency of clotting factor IX (FIX). Hemophilia A is a serious bleeding disorder caused by a deficiency in, or complete absence of, the blood coagulation factor VIII (FVIII) produced by the liver. The clinical presentation for hemophilia A and B is characterized by episodes of spontaneous and prolonged bleeding. There are an estimated 1 in 5,000 and 1 in 20,000 individuals suffering from hemophilia A and B, respectively. Currently, hemophilia A and B is treated with protein replacement therapy using either plasma-derived or recombinant FVIII or FIX. Although protein replacement markedly improved the life expectancy of patients suffering from hemophilia, they are still at risk for severe bleeding episodes and chronic joint damage, since prophylactic treatment is restricted by the short half-life, the limited availability and the high cost of purified clotting factors, which can approach 100.000$/patient/year. In addition, the use of plasma-derived factors obtained from contaminated blood sources increases the risk of viral transmission. Gene therapy offers the promise of a new method of treating hemophilia B, since the therapeutic window is relatively broad and levels slightly above 1% of normal physiologic levels can be therapeutic. If successful, gene therapy could provide a constant FVIII or FIX synthesis in the liver which may lead to a cure for this disease. The different modalities for gene therapy of hemophilia have been extensively reviewed in e.g. Chuah et al., 2012a, 2012b, 2012c; VandenDriessche et al., 2012; High 2001, 2011; and Matrai et al., 2010a, 2010b.

The severity of hemophilia A and hemophilia B has been classified by the subcommittee on Factor VIII and Factor IX of the Scientific and Standardization Committee of the International Society on Thrombosis and Haemostasis into three forms, depending on respectively, the FVIII level and the FIX level: 1) severe form (FVIII or FIX level less than 0.01 international units (IU)/ml, i.e. less than 1% of normal FVIII or FIX level), 2) moderate form (FVIII or FIX level from 0.01 to 0.05 IU/ml, i.e. from 1 to 5% of normal FVIII or FIX level), and 3) mild from (FVIII or FIX level higher than 0.05 to 0.4 IU/ml, i.e. higher than 5 to 40% of normal FVIII or FIX level). Hemophilia A is the most common hereditary coagulation disorder with an incidence approaching approximately 1 in 5000 males.

Protein substitution therapy (PST) with purified or recombinant FVIII and FIX has significantly improved the patients' quality of life. However, PST is not curative and patients are still at risk of developing potentially life-threatening hemorrhages and crippling joint inflammation. Unfortunately, many patients suffering from hemophilia A (up to 40%) develop neutralizing antibodies to FVIII (i.e. “inhibitors”) following PST. Similarly, an estimated 10% of patients suffering from hemophilia B develop “inhibitors” to FIX. These inhibitors complicate the management of bleeding episodes and can render further PST ineffective. These hemophilia patients can be treated with factor VIIa that enables hemostatic correction even in the face of neutralizing antibodies to FVIII or FIX. These limitations of PST, justify the development of gene therapy as a potential alternative for hemophilia treatment. Furthermore, only a modest increase in FIX or FVIII plasma concentration is needed for therapeutic benefit, with levels of more than 1% of normal levels able to achieve markedly reduced rates of spontaneous bleeding and long-term arthropathy.

The liver is the main physiological site of FIX and FVIII synthesis and hence, hepatocytes are well suited target cells for hemophilia gene therapy. From this location, FIX or FVIII protein can easily enter into the blood circulation. Moreover, the hepatic niche may favor the induction of immune tolerance towards the transgene product (Annoni et al., 2007; Follenzi et al., 2004; Brown et al., 2007; Herzog et al., 1999; Matrai et al., 2011; Matsui et al., 2009). Liver-directed gene therapy for hemophilia can be accomplished with different viral vectors including retroviral (Axelrod et al., 1990; Kay et al., 1992; VandenDriessche et al., 1999, Xu et al., 2003, 2005), lentiviral (Ward et al., 2011, Brown et al., 2007, Matrai et al., 2011), adeno-associated viral (AAV) (Herzog et al., 1999) and adenoviral vectors (Brown et al., 2004; Ehrhardt & Kay, 2002). AAV is a naturally occurring replication defective non-pathogenic virus with a single stranded DNA genome. AAV vectors have a favorable safety profile and are capable of achieving persistent transgene expression. Long-term expression is predominantly mediated by episomally retained AAV genomes. More than 90% of the stably transduced vector genomes are extrachromosomal, mostly organized as high-molecular-weight concatamers. Therefore, the risk of insertional oncogenesis is minimal, especially in the context of hemophilia gene therapy where no selective expansion of transduced cells is expected to occur. The major limitation of AAV vectors is the limited packaging capacity of the vector particles (i.e. approximately 5.0 kb, including the AAV inverted terminal repeats), constraining the size of the transgene expression cassette to obtain functional vectors (Jiang et al., 2006). Several immunologically distinct AAV serotypes have been isolated from human and non-human primates (Gao et al., 2002, Gao et al. 2004), although most vectors for hemophilia gene therapy were initially derived from the most prevalent AAV serotype 2. The first clinical success of AAV-based gene therapy for congenital blindness underscores the potential of this gene transfer technology (Bainbridge et al., 2008).

Preclinical studies with the AAV vectors in murine and canine models of hemophilia or non-human primates have demonstrated persistent therapeutic expression, leading to partial or complete correction of the bleeding phenotype in the hemophilic models (Snyder et al., 1997, 1999; Wang et al., 1999, 2000; Mount et al., 2002; Nathwani et al., 2002). Particularly, hepatic transduction conveniently induces immune tolerance to FIX that required induction of regulatory T cells (Tregs) (Mingozzi et al., 2003; Dobrzynski et al., 2006). Long-term correction of the hemophilia phenotype without inhibitor development was achieved in inhibitor-prone null mutation hemophilia B dogs treated with liver-directed AAV2-FIX gene therapy (Mount et al, 2002). In order to further reduce the vector dose, more potent FIX expression cassettes have been developed. This could be accomplished by using stronger promoter/enhancer elements, codon-optimized FIX or self-complementary, double-stranded AAV vectors (scAAV) that overcome one of the limiting steps in AAV transduction (i.e. single-stranded to double-stranded AAV conversion) (McCarty, 2001, 2003; Nathwani et al, 2002, 2006, 2011; Wu et al., 2008). Alternative AAV serotypes could be used (e.g. AAV8 or AAV5) that result in increased transduction into hepatocytes, improve intra-nuclear vector import and may reduce the risk of T cell activation (Gao et al., 2002; Vandenberghe et al., 2006) though it is not certain that this would necessarily also translate to human subjects since the epitopes are conserved between distinct AAV serotypes (Mingozzi et al., 2007). Liver-directed gene therapy for hemophilia B with AAV8 or AAV9 is more efficient than when lentiviral vectors are used, at least in mice, and resulted in less inflammation (VandenDriessche et al., 2007, 2002). Furthermore, studies indicate that mutations of the surface-exposed tyrosine residues allow the vector particles to evade phosphorylation and subsequent ubiquitination and, thus, prevent proteasome-mediated degradation, which resulted in a 10-fold increase in hepatic expression of FIX in mice (Zhong et al., 2008).

These liver-directed preclinical studies paved the way toward the use of AAV vectors for clinical gene therapy in patients suffering from severe hemophilia B. Hepatic delivery of AAV-FIX vectors resulted in transient therapeutic FIX levels (maximum 12% of normal levels) in subjects receiving AAV-FIX by hepatic artery catheterization (Kay et al., 2000). Recently, gene therapy for hemophilia made an important step forward (Nathwani et al., 2012; Nathwani et al., 2014; Commentary by VandenDriessche & Chuah, 2012). Subjects suffering from severe hemophilia B (<1% FIX) were injected intravenously with self-complementary (sc) AAV8 vectors expressing codon-optimized FIX from a liver-specific promoter. FIX expression levels varied between 1% and 6% of normal levels over a period of around 3 years with a vector dose-dependent effect. Notably, all 6 patients in the high dose cohort reached FIX expression levels up to 5% of normal FIX. The FIX expression levels were consistent with a decrease in FIX usage and annual number of bleeding episodes, with the highest relative reduction (i.e. 94%) observed in the highest dose cohort. Nevertheless, subjects were still at risk of bleeding, including trauma-induced bleeds, warranting intermittent FIX coverage, especially in those subjects with lower circulating FIX levels. It is particularly encouraging that the gene therapy was overall well tolerated. The main difference with the previous liver-directed AAV trial is that for the first time sustained therapeutic FIX levels could be achieved after gene therapy. However, the most common study-related adverse event was an asymptomatic elevation in circulating liver enzymes (i.e. alanine aminotransferase, ALT) level, which occurred approximately 7 to 10 weeks after vector infusion in 4 of the 6 patients in the high-dose cohort. This is reminiscent to what has also been observed with AAV2-based vectors in the initial gene therapy clinical trials for hemophilia more than 10 years (Manno et al., 2006). Transient immune suppression using a short course of glucocorticoids was used in an attempt to limit this vector-specific immune response. An identical vector design was used in a more recent clinical trial (ClinicalTrials.gov number: #NCT02396342), although in this case another capsid serotype (i.e. AAV5) was employed to package the therapeutic FIX gene cassette (Miesbach et al., 2018). This was based on the rationale that AAV5 exhibits a more favorably immune profile based on a lower prevalence of neutralizing antibodies (NAbs) compared to AAV2 or AAV8 but is also capable of transducing hepatocytes to a similar extent as AAV8, at least in non-human primates. However, comprehensive studies are needed using standardized validated assays to better appreciate the global seroprevalence of different AAV serotypes, since regional and specific population effects can influence the read-outs. One previous AAV5-based clinical trial suggests that AAV5 does not appear to elicit a cellular immune responses against the capsid (D'Avola et al., 2015). Nevertheless, it would seem somewhat premature to draw any definitive conclusions about the possible immune ramifications of using AAV5 over AAV8 based on the relatively limited number of patients involved.

Ten adults with severe hemophilia B were included in this open-label clinical study and none had pre-existing anti-AAV5 neutralizing antibodies. Study participants were enrolled in low (5×10¹² vg/kg) and high (2×10¹³ vg/kg) vector dose cohorts. Mean FIX expression levels corresponding to 4.4% were achieved in the patients in the low dose cohorts increasing to 6.9% in the high dose cohort. This was consistent with a reduction in FIX usage and annualized spontaneous bleeding rate, though the risk of trauma-induced bleeds remained high as before the gene therapy intervention. Interestingly, prophylaxis was no longer required in eight of 9 patients who received prophylactic treatment before gene therapy. FIX expression was sustained for one year and for about 6 months in the low and high dose cohorts, respectively. Asymptomatic and transient liver transaminase elevations were detected in 3 of the trial participants but this did not seem to correlate with any AAV capsid-specific T-cell responses or decrease in FIX activity. The study demonstrates that changing serotype from AAV5 to AAV8 does not prevent this adverse event in AAV-based gene therapy. Consequently, patients were given transient immunosuppressive treatment with glucocorticoids to block these unwanted immune responses and transaminase elevations.

One of the significant limitations in the generation of efficient viral gene delivery systems for the treatment of hemophilia A by gene therapy is the large size of the FVIII cDNA. Previous viral vector-based gene therapy studies for hemophilia A typically relied on the use of small but weak promoters, required excessively high vector doses that were not clinically relevant or resulted in severely compromised vector titers. Several other ad hoc strategies were explored, such as the use of split or dual vector design to overcome the packaging constraints of AAV, but these approaches were overall relatively inefficient and raised additional immunogenicity concerns (reviewed in Petrus et al., 2010). It has been found that the FVIII B domain is dispensable for procoagulant activity. Consequently, FVIII constructs in which the B domain is deleted are used for gene transfer purposes since their smaller size is more easily incorporated into vectors. Furthermore, it has been shown that deletion of the B domain leads to a 17-fold increase in mRNA and primary translation product. FVIII wherein the B domain is deleted and replaced by a short 14-amino acid linker is currently produced as a recombinant product and marketed as Refacto® for clinical use (Wyeth Pharma) (Sandberg et al., 2001). Miao et al. (2004) added back a short B domain sequence to a B domain deleted FVIII, optimally 226 amino acids and retaining 6 sites for N-linked glycosylation, to improve secretion. McIntosh et al. (2013) replaced the 226 amino-acid spacer of Miao et al. with a 17 amino-acid peptide in which six glycosylation triplets from the B-domain were juxtaposed. Yet, production was still not sufficient for therapeutic purposes. Also codon optimization of human factor VIII cDNAs leads to high-level expression. Significantly greater levels (up to a 44-fold increase and in excess of 200% normal human levels) of active FVIII protein were detected in the plasma of neonatal hemophilia A mice transduced with lentiviral vector expressing FVIII from a codon-optimized cDNA sequence, thereby successfully correcting the disease model (Ward et al., 2011).

An exemplary state of the art vector for liver-specific expression of FIX is described in WO 2009/130208 and is composed of a single-stranded AAV vector that contains the TTR/Serp regulatory sequences driving a factor cDNA. A FIX first intron was included in the vector, together with a polyadenylation signal. Using said improved vector yielded about 25-30% stable circulating factor IX.

In order to translate viral-vector based gene therapy for hemophilia to the clinic, the safety concerns associated with administering large vector doses to the liver and the need for manufacturing large amounts of clinical-grade vector must be addressed. Increasing the potency (efficacy per dose) of gene transfer vectors is crucial towards achieving these goals. It would allow using lower doses to obtain therapeutic benefit, thus reducing potential toxicities and immune activation associated with in vivo administration, and easing manufacturing needs.

One way to increase potency is to engineer the transgene sequence itself to maximize expression and biological activity per vector copy. Present inventors have previously shown that FIX transgenes optimized for codon usage and carrying an R338L amino acid substitution associated with clotting hyperactivity and thrombophilia (Simioni et al., 2009), increase the efficacy of gene therapy using lentiviral vectors or AAV vectors up to 15-fold in hemophilia B mice, without detectable adverse effects, substantially reducing the dose requirement for reaching therapeutic efficacy and thus facilitating future scale up and its clinical translation (Cantore et al., 2012, Nair et al., 2014).

Two independent recent gene therapy clinical trials had been conducted based on the FIX-R338L Padua variant showing promising results. In addition, several other trials are in the pipeline that have strategically moved their focus away from using the wild-type FIX to the hyperactive FIX-R338L variant instead suggesting that it is now becoming the gold standard for hemophilia B gene therapy. In the first FIX-R338L Padua trial (Baxalta, now part of Shire; NCT01687608; see also Monahan et al. (2015) and Horling et al. (2017)), severe hemophilia B patients were treated by a single intravenous injection with a self-complementary (sc) AAV8 vector expressing a codon-optimized FIX-R338L Padua variant (designated as BAX335). In this trial 3 dosing cohorts were performed, treating 7 patients in total. Two of the patients showed transient FIX activity levels of more than 50% than eventually declined to basal levels. One of the patients from the medium dose cohort (10¹² vg/kg) showed persistent FIX levels for more than a year in the 20% activity range. However, FIX expression was either not detectable or not sustained in the majority of the patients enrolled in this trial. A root cause analysis suggested that the potential cause for decreased FIX expression could be due to exaggerated immunogenicity. Indeed, BAX335 contains a high number of CpG dinucleotide motifs in the FIX coding sequence which may have contributed to increased immunogenicity by stimulating the innate immune system in a Toll-like Receptor 9 (TLR9)-dependent fashion (Faust et al., 2013).

The second FIX-R338L Padua trial (Spark Therapeutics/Pfizer; NCT02484092) resulted in a more consistent response in the patients compared to the outcome of the BAX335 trial. A single-stranded AAV vector (designated as SPK-9001) was designed that expressed a codon-optimized FIX-F338L Padua variant from a hepatocyte-specific promoter composed of the apolipoprotein E gene hepatic-control region (APOE) and a liver-specific human al-antitrypsin (hAAT) promoter (George et al., 2017). The AAV vector was packaged using an alternative mutated AAV capsid (designated as AAV-Spark100) based on its favorable seropositivity profile. A relatively low dose of vectors (5×10¹¹ vg/kg) was injected intravenously in 10 patients with severe hemophilia B. Fourteen weeks after vector administration, the 10 participants reached a steady-state FIX activity level of around 33.7±18.5% consistent with a decrease in both annualized bleeding rate (from 11.1 to 0.4 bleeding events/year) and number of infusions per year (from 67.5 to 1.2). FIX activity was sustained for 1 year after a single intravenous injection of the gene therapy vector. This prompted discontinuation of prophylaxis by protein substitution therapy. As in the case of all the other hemophilia B gene therapy trials, here also two of the trial participants showed a transient elevation of liver transaminases that coincided with an AAV capsid-specific T cell immune response, requiring treatment with tapering doses of oral corticosteroids. In one of the trial participants that was treated by transient immune suppression, FIX expression was stable in the 70-90% range of normal FIX activity. However, in the second patient, immune suppression did not suffice to prevent a significant reduction in FIX levels. This suggests that it may perhaps be necessary and prudent to administer oral corticosteroids much earlier as a prophylactic treatment before the onset of transaminitis.

Based on encouraging results in hemophilic mice and non-human primates (Bunting et al., 2018), 9 patients with severe hemophilia A were injected intravenously with an AAV5 vector encoding a codon-optimized B-domain—deleted human factor VIII (AAV5-hFVIII-SQ) (Rangarajan et al., 2017). At the low (1 patient; 6×10¹² vg/kg) and intermediate dose (1 patient; 2×10¹³ vg/kg), no circulating FVIII levels were detected. However, 6 of 7 patients treated with the highest vector dose of 6×10¹³ vg/kg showed a significant increase in FVIII activity levels that reached more than 50% of normal FVIII activity levels after 20 weeks. The vector doses used in this trial appear to be substantially higher than the doses used in any of the AAV-based hemophilia B trials. Nevertheless, in the absence of any standards, some caution is warranted to compare vector doses and trial outcomes. Most importantly, one year after vector injection, a mean FVIII activity level of 93±48% was achieved in the trial participants who received the highest dose. However, in four of the patients, levels of more than 150% of normal FVIII activity levels were attained, with peaks ranging from 201 to 349% of normal. This raised some concerns regarding possible increased thrombotic risk but these supra-physiologic levels were not sustained and at 78 weeks FVIII levels further declined and fell within the physiologic range. The six patients of the highest dose that were previously on FVIII prophylaxis showed a reduction in annualized bleeding rate from 16 to 1 event per year after gene therapy. Similarly, the median annualized FVIII infusion rate dropped from 138 infusions per year to 2 after gene therapy. As in the hemophilia B trials, some of the patients in the high-dose cohort showed a significant increase in transaminase levels, requiring tapering doses of glucocorticoid treatment. This suggests that AAV5-based gene therapy resulted in liver inflammation though there was no evidence of a T-cell-mediated immune responses to the AAV5 capsid. This is reminiscent of the results obtained in the AAV5-based hemophilia B trial, described above (Miesbach et al., 2018).

In WO2014/064277 expression vectors are described which combine the robust Serpin enhancer with codon-optimized transgenes encoding FIX or FVIII, resulting in increased liver-specific expression of FIX and FVIII, respectively.

However, the limit of what could be achieved with strategies such as promoter engineering and codon-optimisation of transgenes in terms of maximizing gene expression could be reached. Recent successes in protein engineering demonstrated that fusing the FIX protein with either albumin or immunoglobulin Fc domains can significantly prolong the half-live of the FIX protein (Peters et al., 2010; Powell et al., 2013; Metzner et al., 2009; Santagostino et al., 2016). Consequently, hemophilia B patients require far less frequent infusions with these longer-acting factors than by conventional protein substitution therapy with standard recombinant FIX proteins to maintain adequate hemostasis.

It is an object of the present invention to further increase the efficiency and safety of liver-directed gene therapy for hemophilia A and B.

### SUMMARY OF THE INVENTION

While it was previously shown that fusing the FIX protein with albumin significantly prolongs the half-live of the FIX protein (Metzner et al., 2009; Santagostino et al., 2016), these studies relate to the production of recombinatent FIX-Alb fusion proteins and the administration of said recombinant FIX-Alb fusion proteins (and not vectors) to a subject. The present inventors found that when administering viral vectors expressing the prior art, non codon-optimised, fusion gene encoding the FIX and human albumin fusion protein, there was no significant difference in circulating levels and/or activity of the fusion protein and no significant expression level increase was detected versus the viral vector expressing the FIX transgene alone. This indicates that the success reported using recombinant fusion genes with human albumin could not be repeated using viral vectors for gene therapy purposes. However, when administering a vector expressing a fusion gene comprising a codon optimised human albumin according to SEQ ID NO: 14 and the FIX transgene, encoding a FIX-human albumin fusion protein, a significant increase in the gene expression of the fusion gene and in the circulating levels and activity of the FIX protein was observed.

The invention hence provides for the following aspects:

Aspect 1. A codon-optimised nucleic acid molecule encoding human albumin comprising a sequence defined by SEQ ID NO: 14 or a sequence having at least 80% sequence identity to said sequence, preferably a sequence defined by SEQ ID NO: 14.

Aspect 2. The nucleic acid molecule according to aspect 1, comprising a transgene fused to said sequence defined by SEQ ID NO: 14 or said sequence having at least 80% sequence identity to said sequence, preferably wherein said transgene is a codon optimized transgene.

Aspect 3. The nucleic acid molecule according to aspect 1 or 2, wherein said transgene is located at the 5′ end of said sequence defined by SEQ ID NO: 14 or said sequence having at least 80% sequence identity to said sequence.

Aspect 4. The nucleic acid molecule according to any one of aspects 1 to 3, wherein said transgene is separated from said sequence defined by SEQ ID NO: 14 or said sequence having at least 80% sequence identity to said sequence by a sequence encoding one or more polypeptide or peptide linkers, preferably by a peptide linker defined by SEQ ID NO: 18.

Aspect 5. The nucleic acid molecule according to any one of claims 1 to 4 for use in increasing the expression and/or circulation level and/or activity of a protein or polypeptide encoded by a transgene.

Aspect 6. A nucleic acid expression cassette comprising the nucleic acid molecule according to any one of aspects 1 to 4, operably linked to a promoter.

Aspect 7. The nucleic acid expression cassette according to aspect 6, comprising at least one tissue-specific nucleic acid regulatory element operably linked to the promoter and the nucleic acid molecule as defined in any one of aspects 1 to 5.

Aspect 8. The nucleic acid expression cassette according to aspect 6 or 7, comprising a minute virus of mice (MVM) intron, preferably the MVM intron defined by SEQ ID NO: 20.

Aspect 9. The nucleic acid expression cassette according to any one of aspects 6 to 8, comprising a transcriptional termination signal, preferably a polyadenylation signal, more preferably a synthetic polyadenylation signal defined by SEQ ID NO: 21 or the Simian Virus 40 (SV40) polyadenylation signal defined by SEQ ID NO: 23.

Aspect 10. The nucleic acid expression cassette according to any one of aspects 6 to 9, wherein said transgene encodes a secretable therapeutic protein or a secretable immunogenic protein, preferably the transgene encodes for a secretable therapeutic protein selected from the list consisting of: factor IX, factor VIIa, factor VIII, hepatocyte growth factor (HGF), tissue factor (TF), tissue factor pathway inhibitor (TFPI), ADAMTS13, vascular endothelial growth factor (VEGF), placental growth factor (PLGF), fibroblast growth factor (FGF), soluble fms-like tyrosine kinas1 (sFLT1), α1-antitrypsin (AAT), insulin, proinsulin, factor VII, factor X, von Willebrand factor, C1 esterase inhibitor (C1-INH), lysosomal enzymes, lysosomal enzyme iduronate-2-sulfatase (I2S), erythropoietin (EPO), interferon-α, interferon-β, interferon-γ, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C-X-C motif) ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1), tumor necrosis factor (TNF), afamin (AFM), α1-antitrypsin, α-galactosidase A, α-L-iduronidase, lipoprotein lipase, apoliproteins, low-density lipoprotein receptor (LDL-R), albumin, dipeptidyl peptidase (DPP-4)-resistant glucagon-like peptide 1 (GLP-1), GLP-2, glucagon, growth hormone (GH), interferons (e.g. IFNalpha-2b), β-natriuretic peptide, IL-1Ra, exendin-4, oxyntomodulin, follistatin, antibodies and nanobodies.

Aspect 11. The nucleic acid expression cassette according to aspect 10, wherein said transgene encodes a therapeutic protein for treating and/or preventing liver-related disorders, preferably hemophilia A, hemophilia B or factor VII deficiency.

Aspect 12. The nucleic acid expression cassette according to aspect 11, wherein said transgene encodes for coagulation factor IX (FIX), preferably wherein said coagulation factor FIX contains a hyper-activating mutation, more preferably wherein said hyper-activating mutation corresponds to an R338L amino acid substitution, more preferably wherein said transgene encodes for coagulation factor IX having a nucleic acid sequence defined by SEQ ID NO: 11.

Aspect 13. The nucleic acid expression cassette according to aspect 12, wherein said transgene encodes for coagulation factor VIII (FVIII), preferably wherein said transgene is codon-optimized coagulation factor FVIII, or wherein said coagulation factor VIII has a deletion of the B domain, preferably wherein said B domain of said FVIII is replaced by a linker defined by SEQ ID NO: 15, more preferably wherein said transgene encodes for coagulation factor VIII having a nucleic acid sequence defined by SEQ ID NO: 16.

Aspect 14. The nucleic acid expression cassette according to aspect 12, wherein said transgene encodes for the light chain and the heavy chain of coagulation factor VII (FVII) or factor FVIIa (FVIIa), wherein the light chain of FVII is coupled to the heavy chain of FVII or FVIIa by one or more cleavable polypeptide or peptide linkers, preferably wherein said transgene encodes for an amino acid sequence as defined by SEQ ID NO: 34.

Aspect 15. The nucleic acid expression cassette according to any one of aspects 11 to 14, wherein the at least one tissue-specific nucleic acid regulatory element is at least one liver-specific nucleic acid regulatory element.

Aspect 16. The nucleic acid expression cassette according to aspect 15, wherein the at least one liver-specific nucleic acid regulatory element comprises the Serpin enhancer defined by SEQ ID NO: 25 or a sequence having at least 95% identity to said sequence.

Aspect 17. The nucleic acid expression cassette according to aspect 16, comprising a triple repeat, preferably tandemly arranged, of the Serpin enhancer defined by SEQ ID NO: 25 or the sequence having at least 95% identity to said sequence.

Aspect 18. The nucleic acid expression cassette according to any one of aspects 11 to 17, wherein the promoter is a liver-specific promoter, preferably a liver-specific promoter is selected from the group comprising: the transthyretin (TTR) promoter, the minimal TTR promotor (TTRm), the AAT promoter, the albumin (ALB) promotor or minimal promoter, the apolipoprotein A1 (APOA1) promoter or minimal promoter, the complement factor B (CFB) promoter, the ketohexokinase (KHK) promoter, the hemopexin (HPX) promoter or minimal promoter, the nicotinamide Nmethyltransferase (NNMT) promoter or minimal promoter, the (liver) carboxylesterase 1 (CES1) promoter or minimal promoter, the protein C (PROC) promoter or minimal promoter, the apolipoprotein C3 (APOC3) promoter or minimal promoter, the mannan-binding lectin serine protease 2 (MASP2) promoter or minimal promoter, the hepcidin antimicrobial peptide (HAMP) promoter or minimal promoter, or the serpin peptidase inhibitor, clade C (antithrombin), member 1 (SERPINC1) promoter or minimal promoter, preferably the TTRm.

Aspect 19. A vector comprising the nucleic acid expression cassette according to any one of aspects 6 to 18, preferably wherein said vector is a viral vector, more preferably wherein said vector is derived from an adeno-associated virus (AAV).

Aspect 20. The vector according to aspect 19, wherein said vector is a single-stranded AAV.

Aspect 21. The vector according to aspect 20, having SEQ ID NO: 2, SEQ ID NO: 3, SEQ ID NO: 6 or SEQ ID NO: 8, preferably SEQ ID NO: 8.

Aspect 22. A pharmaceutical composition comprising the vector according to any one of aspects 19 to 21, and a pharmaceutically acceptable carrier.

Aspect 23. The vector according to any one of aspects 19 to 21 or the pharmaceutical composition according to aspect 22 for use in medicine, preferably gene therapy, more preferably liver-directed gene therapy.

Aspect 24. The vector according to any one of aspects 19 to 21 or the pharmaceutical composition according to aspect 22 for use in the treatment of a liver-related disorder, preferably hemophilia A, hemophilia B or FVII deficiency if the transgene is FIX (hemophilia B), FVIII (hemophilia A) or FVII (hemophilia A, hemophilia B, preferably patients with inhibitors to FVIII or FIX; FVII deficiency).

Aspect 25. A method of treating a liver-related disorder, preferably hemophilia, in a subject in need of such a treatment, comprising administering a therapeutically effective amount of vector according to any one of aspects 19 to 21 or the pharmaceutical composition according to aspect 22 to the subject.

Aspect 26. Use of the vector according to any one of aspects 19 to 21 or the pharmaceutical composition according to aspect 22 for the manufacture of a medicament for the treatment of a liver-related disorder, preferably hemophilia, in a subject.

Aspect 27. An in vitro or ex vivo method for expressing a transgene product in liver cells comprising:
introducing the nucleic acid expression cassette according to any one of aspects 6 to 18, or the vector according to any one of aspects 19 to 21 into the liver cells;
expressing the transgene product in the liver cells.

Aspect 28. Use of the nucleic acid molecule according to any one of aspects 1 to 4, the nucleic acid expression cassette according to any one of aspects 6 to 18 or the vector according to anyone of aspects 19 to 21 for increasing the expression and/or circulation level and/or activity of a protein or polypeptide encoded by a transgene, preferably wherein said use is an in vitro use.

Aspect 29. A method for increasing the expression and/or circulation level and/or activity of a protein or polypeptide encoded by a transgene using the nucleic acid molecule according to any one of aspects 1 to 4, the nucleic acid expression cassette according to any one of aspects 6 to 18 or the vector according to anyone of aspects 19 to 21.

### BRIEF DESCRIPTION OF THE FIGURES

The present invention is illustrated by the following figures which are to be considered for illustrative purposes only and in no way limit the invention to the embodiments disclosed therein:

FIG. 1: Plasmid map of the pAAVss-1XSERP-mTTR-MVM-hFIXco-SV40pA vector

FIG. 2: Plasmid map of the pAAVss-1XSERP-mTTR-MVM-hFIXco-Alb-SV40pA vector

FIG. 3: Plasmid map of the pAAVss-1XSERP-mTTR-MVM-hFIXco-Albco-SV40pA vector

FIG. 4: Plasmid map of the pAAVss-1XSERP-mTTR-MVM-hFIXcoPadua-SV40pA vector

FIG. 5: Plasmid map of the pAAVss-1XSERP-mTTR-MVM-hFIXcoPadua-Alb-SV40pA vector

FIG. 6: Plasmid map of the pAAVss-1XSERP-mTTR-MVM-hFIXcoPadua-Albco-SV40pA vector

FIG. 7: Plasmid map of the pAAVss-3XSERP-mTTR-MVM-hFIXcoPadua-Alb-SV40pA vector

FIG. 8: Plasmid map of the pAAVss-3XSERP-mTTR-MVM-hFIXcoPadua-Albco-SV40pA vector

FIG. 9: FIX protein levels and activity upon transduction of the described vectors in FIX knock-out (KO) mice. 9A): FIX protein expression levels achieved upon transduction with 5×10⁹ vg/mouse over time for AAVss-1XSERP-mTTR-MVM-hFIXcoPadua-SV40pA and AAVss-1XSERP-mTTR-MVM-hFIXcoPadua-Alb-SV40pA over time; 9B): FIX protein activity achieved upon transduction with 5×10⁹ vg/mouse for AAVss-1XSERP-mTTR-MVM-hFIXcoPadua-SV40pA and AAVss-1XSERP-mTTR-MVM-hFIXcoPadua-Alb-SV40pA over time; 9C) the protein activity achieved upon transduction with 5×10⁹ vg/mouse for AAVss-1XSERP-mTTR-MVM-hFIXcoPadua-SV40pA and AAVss-1XSERP-mTTR-MVM-hFIXcoPadua-Albco-SV40pA over time.

FIG. 10: FIX protein levels and mRNA expression upon transduction of the described vectors in mice. 10A): FIX protein expression levels achieved upon transduction with 1×10⁹ vg/mouse over time for AAVss-1XSERP-mTTR-MVM-hFIXco-SV40pA, AAVss-1XSERP-mTTR-MVM-hFIXco-Albco-SV40pA and AAVss-3XSERP-mTTR-MVM-hFIXco-Albco-SV40pA over time; 10B): the mRNA expression relative to control achieved upon transduction with 1×10⁹ vg/mouse for AAVss-1XSERP-mTTR-MVM-hFIXco-SV40pA, AAVss-1XSERP-mTTR-MVM-hFIXco-Albco-SV40pA and AAVss-3XSERP-mTTR-MVM-hFIXco-Albco-SV40pA.

FIG. 11: FIX protein and activity levels upon transduction of the described vectors in FIX knock-out (KO) mice. 11A): the protein expression levels achieved upon transduction with 5×10⁹ vg/mouse over time for AAVss-3XSERP-mTTR-MVM-hFIXcoPadua-Alb-SV40pA and AAVss-3XSERP-mTTR-MVM-hFIXcoPadua-Albco-SV40pA over time; 11B): the protein activity achieved upon transduction with 5×10⁹ vg/mouse for AAVss-3XSERP-mTTR-MVM-hFIXcoPadua-Alb-SV40pA and AAVss-3XSERP-mTTR-MVM-hFIXcoPadua-Albco-SV40pA over time.

FIG. 12: FIX antigen levels and FIX activity levels achieved upon transduction of the described vectors in FIX knock-out (KO) mice with 5×10⁸ vg/mouse, 1×10⁹ vg/mouse, or 5×10⁹ vg/mouse over time (1 and 3 weeks).

FIG. 13: FIX activity levels achieved upon transduction of the described vectors in FIX knock-out (KO) mice with 5×10⁸ vg/mouse (A), 1×10⁹ vg/mouse (B; D), or 5×10⁹ vg/mouse (C; E) over time (expressed in weeks post injection).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

The terms or definitions provided herein are to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art.

The definitions provided herein should not be construed to have a scope less than understood by a person of ordinary skill in the art.

The present inventors have unexpectedly found that codon optimised human albumin (also called “Albco” herein), preferably codon optimised human albumin having a sequence defined by SEQ ID NO: 14 or a sequence having at least 80% sequence identity, preferably at least 85% sequence identity, to said sequence, can be used to enhance gene expression of a transgene and/or to increase the levels and/or activity of a protein or polypeptide encoded by a transgene, when genetically fused to said transgene. More particularly, codon optimised human albumin can be used to prepare genetically fused transgenes encoding e.g. human coagulation factor IX (hFIX)-Alb, human coagulation factor VIII (hFVIII)-Alb or human coagulation factor FVII-Alb, (also called “fusion genes” herein) to enhance gene expression of hFIX, hFVIII or hFVII, respectively, and/or to increase the expression or circulation levels and/or activity of hFIX, hFVIII or hFVII, respectively, in vitro and in vivo. In contrast, the beneficial effects on gene expression and/or levels and/or activity of a protein or polypeptide encoded by a transgene are not observed if non-codon optimised (also called “wild-type” herein) human albumin (also called “Alb” herein) is genetically fused to said transgene.

Accordingly, a first aspect provides a codon-optimised nucleic acid molecule encoding human albumin for use in enhancing gene expression of a transgene and/or for increasing the levels and/or activity of a protein or polypeptide encoded by a transgene, said nucleic acid molecule comprising a sequence defined by SEQ ID NO: 14 or a sequence having at least 80% sequence identity to said sequence, preferably comprising a sequence defined by SEQ ID NO: 14.

The term “nucleic acid molecule” or “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g., chemically synthesised) DNA, RNA or DNA/RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear. Such nucleic acid molecule or nucleic acid may be suitably isolated.

A nucleotide sequence defined by SEQ ID NO: 14 represents a codon optimised form of wild-type, or non-codon optimized, human albumin cDNA as defined in SEQ ID NO: 28 and of which the precursor form thereof is annotated under NCBI Genbank (http://www.ncbi.nlm.nih.gov/) accession number NM_000477.6.

The human albumin protein encoded by the codon-optimised nucleic acid molecule as taught herein may be wild-type human albumin protein as defined in SEQ ID NO: 27 and of which the amino acid sequence of the precursor form is annotated under NCBI Reference sequence NP_000468.1, or may be a variant or mutant thereof which carries amino acid sequence variations vis-à-vis the corresponding native protein, such as, e.g., amino acid deletions, additions and/or substitutions. For example, human albumin may also encompass the K573P mutation (i.e. wherein the lysine at amino acid residue position 573 is substituted by proline) of albumin as described in Andersen et al., 2014 and Strohl et al., 2015.

The term “codon optimised” when used in relation with a transgene refers to modifying the codons of a transgene without altering the amino acid sequence of the protein or polypeptide encoded by said transgene. Typically, rare codons in the transgene (i.e. codons that are rarely used in the host in which the transgene is to be expressed) are replaced by codons that are more abundant in the transgenes of the host organism.

The term “codon” as used herein refers to any group of three consecutive nucleotide bases in a given messenger RNA (mRNA) molecule, or coding DNA (cDNA) encoding a particular amino acid residue in a protein or polypeptide or for the termination of translation (“stop codon”). The term “codon” also encompasses base triplets in a DNA strand. In silico prediction of codon-optimized transgenes may be obtained by any method known in the art, for example using commercial codon optimisation tools such as the OptimumGene™ Gene Design system (GenScript) or Codon Optimization Tool (OMICS_23398), Omictools). Of course these are only predictive tools and the actual effect of each codon-optimisation remains uncertain and requires extensive testing.

The skilled person will understand that limited variations (e.g. one or more nucleotide additions, deletions, or substitutions relative to (i.e., compared with) the corresponding nucleic acid) in the nucleotide sequence defined by SEQ ID NO: 14 may still result in a nucleotide sequence encoding for the identical protein as the nucleotide sequence defined by SEQ ID NO: 14 as described herein.

Accordingly, in particular embodiments, the nucleic acid molecule as taught herein comprises a sequence defined by SEQ ID NO: 14 or a sequence being at least about 80% identical, e.g. preferably at least about 85% identical, e.g., more preferably at least about 90% identical, e.g., at least 91% identical, 92% identical, even more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical to SEQ ID NO: 14.

As used herein, the terms “identity” and “identical” and the like refer to the sequence similarity between two polymeric molecules, e.g., between two nucleic acid molecules, e.g., two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250). Typically, the percentage sequence identity is calculated over the entire length of the sequence. As used herein, the term “substantially identical” denotes at least 90%, preferably at least 95%, such as 95%, 96%, 97%, 98% or 99%, sequence identity.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

Such protein, polypeptide or peptide may be suitably isolated. The term “isolated” with reference to a particular component (such as for instance a nucleic acid, protein, polypeptide or peptide) generally denotes that such component exists in separation from—for example, has been separated from or prepared and/or maintained in separation from—one or more other components of its natural environment. For instance, an isolated human or animal protein or complex may exist in separation from a human or animal body where it naturally occurs.

In particular embodiments, the nucleic acid molecule as taught herein comprises a transgene fused to said sequence defined by SEQ ID NO: 14 or said sequence having at least 80% sequence identity to said sequence, preferably wherein said transgene is a codon optimized transgene.

In the context of present invention, the term “fused” as used herein is synonymous with “connected”, “bound”, “coupled”, “joined” and refers to a physical link between at least two elements or components. In the context of two genes or protein encoding nucleotide sequences, “fused” refers to fusion of the coding sequences (called “genetic fusion”), resulting in a fusion protein upon expression.

Fusion of the transgene to the sequence defined by SEQ ID NO: 14 or said sequence having at least 80% sequence identity to said sequence typically results in the formation of a fusion gene or chimeric gene encoding a fusion protein or polypeptide. The terms “fusion protein” or “fusion polypeptide” denote the product of genetic fusions, whereby two or more proteins, polypeptides or variants or fragments thereof are joined by a co-linear, covalent linkage via their individual polypeptide backbones, through genetic expression of a single contiguous polynucleotide molecule encoding the fusion product. Typically, to produce the contiguous polynucleotide molecule encoding the fusion product, two or more open reading frames (ORFs) each encoding a given polypeptide segment are joined to form a continuous longer ORF in a manner that maintains the correct reading frame for each original ORF. In the resulting recombinant fusion polypeptide the two or more polypeptide segments encoded by the original ORFs are joined in the same polypeptide molecule, whereas they are not normally so joined in nature. While the reading frame is thus made continuous throughout the fused genetic segments, the so fused polypeptide segments may be physically or spatially separated by, for example, an in-frame polypeptide or peptide linker, which may or may not be cleavable.

The skilled person will understand that depending on the order of the transgene and the sequence defined by SEQ ID NO: 14 or said sequence having at least 80% sequence identity to said sequence, the stop codon of the most upstream positioned sequence may need to be removed to avoid truncation of the fusion protein or polypeptide encoded by the fusion gene. Furthermore, the skilled person will also understand that the transgene and the sequence defined by SEQ ID NO: 14 or said sequence having at least 80% sequence identity to said sequence will need to be in—frame for the protein or polypeptide encoded by the fusion gene to be effectively made.

The term “transgene” or “(trans)gene” as used herein refers to particular nucleic acid sequences encoding a polypeptide or a portion of a polypeptide to be expressed in a cell into which the nucleic acid sequence is inserted. However, it is also possible that transgenes are expressed as RNA, typically to lower the amount of a particular polypeptide in a cell into which the nucleic acid sequence is inserted. These RNA molecules include but are not limited to molecules that exert their function through RNA interference (shRNA, RNAi), micro-RNA regulation (miRNA), catalytic RNA, antisense RNA, RNA aptamers, etc. How the nucleic acid sequence is introduced into a cell is not essential to the invention, it may for instance be through integration in the genome or as an episomal plasmid, or by means of a viral or non-viral vector. Of note, expression of the transgene may be restricted to a subset of the cells into which the nucleic acid sequence is inserted. The term ‘transgene’ is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been introduced; (3) a nucleic acid sequence that serves to add additional copies of the same (i.e., homologous) or a similar nucleic acid sequence naturally occurring in the cell into which it has been introduced; or (4) a silent naturally occurring or homologous nucleic acid sequence whose expression is induced in the cell into which it has been introduced. By ‘mutant form’ is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

In particular embodiments, the transgene is a codon optimized transgene.

In particular embodiments, the transgene encodes for a secretable protein.

In particular embodiments, the transgene encodes for a therapeutic protein or an immunogenic protein, preferably a secretable therapeutic protein or a secretable immunogenic protein.

The term “secretable protein” as used herein refers to proteins that are expressed in specific cells or a specific tissue, such as the liver, and that are then exported to the blood stream for transport to other portions of the body.

In embodiments, the transgene encodes a secretable therapeutic protein, such as hormones, cytokines, chemokines, growth factors, exoenzymes (e.g. glucosidase, lipoprotein lipase, alpha1-antitrypsin), plasma factors, clotting factors, erythropoietin, antibodies and nanobodies.

Non-limiting examples of secretable therapeutic proteins include factor IX, factor VIII, factor VII, factor VIIa (FVIIa), hepatocyte growth factor (HGF), tissue factor (TF), tissue factor pathway inhibitor (TFPI), ADAMTS13, vascular endothelial growth factor (VEGF), placental growth factor (PLGF), fibroblast growth factor (FGF), soluble fms-like tyrosine kinas1 (sFLT1), α1-antitrypsin (AAT), insulin, proinsulin, factor X, von Willebrand factor, C1 esterase inhibitor (C1-INH), lysosomal enzymes, lysosomal enzyme iduronate-2-sulfatase (I2S), erythropoietin (EPO), interferon-α, interferon-β, interferon-γ, interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), chemokine (C-X-C motif) ligand 5 (CXCL5), granulocyte-colony stimulating factor (G-CSF), granulocyte-macrophage colony stimulating factor (GM-CSF), macrophage colony stimulating factor (M-CSF), stem cell factor (SCF), keratinocyte growth factor (KGF), monocyte chemoattractant protein-1 (MCP-1), tumor necrosis factor (TNF), afamin (AFM), alpha1-antitrypsin, alpha-galactosidase A, alpha-L-iduronidase, lipoprotein lipase, apoliproteins (e.g. apolipoprotein A-I (apoA-I)), low-density lipoprotein receptor (LDL-R), albumin, dipeptidyl peptidase (DPP-4)-resistant glucagon-like peptide 1 (GLP-1), GLP-2, glucagon, growth hormone (GH), interferons (e.g. IFNalpha-2b), β-natriuretic peptide, IL-1Ra, exendin-4, oxyntomodulin, follistatin, transgenes encoding antibodies, nanobodies, and fragments, subunits or mutants thereof, etc.

In embodiments, the transgene encodes a secretable immunogenic protein. Non-limiting examples of secretable immunogenic proteins or subunits include antigens derived from cancer cells (HER2), viruses (HPV, HBV), bacteria (pertussis, diphtheria, tetanus) and fungi or parasites (malaria).

As used herein, the term “immunogenic” refers to a substance or composition capable of eliciting an immune response.

In particular embodiments, said transgene encodes a secretable therapeutic protein for treating and/or preventing liver-related disorders, preferably hemophilia.

Typically, the transgenes in the nucleic acid molecules, expression cassettes and vectors described herein encode coagulation factor IX, coagulation factor VIII or coagulation factor VII (or coagulation factor VIIa) preferably human coagulation factor IX, coagulation factor VIII or coagulation factor VII (or coagulation factor VIIa), more preferably human coagulation factor IX.

The term “coagulation factor IX” has the meaning as known in the art. Synonyms of coagulation factor IX are “FIX” or “Christmas factor” or “F9” and can be used interchangeably. In particular, the term “coagulation factor IX” encompasses the human protein encoded by the mRNA sequence as defined in Genbank accession number NM_000133.

Preferably, said FIX is a mutated FIX, which is hyperactive or hyper-functional as compared to the wild type FIX. Modifying functional activity of human coagulation factor can be done by bioengineering e.g. by introduction of point mutations. By this approach a hyperactive R338A variant was reported, which showed a 3 fold increased clotting activity compared to the wild type human FIX in an in vitro activated partial thromboplastin time assay (APPT) (Chang et al., 1998) and a 2 to 6-fold higher specific activity in hemophilia B mice transduced with the mutant FIX gene (Schuettrumpf et al., 2005). Further exemplary FIX point-mutants or domain exchange mutants with even higher clotting activities have been described: FIX, with the EGF-1 domain replaced with the EGF-1 domain from FVII, alone or in combination with a R338A point mutation (Brunetti-Pierri et al., 2009), the V86A/E277A/R338A triple mutant (Lin et al., 2010), the Y259F, K265T, and/or Y345T single, double or triple mutants (Milanov, et al., 2012), and the G190V point mutant (Kao et al., 2010), all incorporated herein by reference. In a particularly preferred embodiment, the FIX mutant is the one described by Simioni et al., in 2009 and denominated as the “factor IX Padua” mutant, causing X-linked thrombophilia. Said mutant factor IX is hyperactive and carries an R338L amino acid substitution. In a preferred embodiment of the present invention, the FIX transgene used in nucleic acid expression cassettes and expression vectors described herein encodes the human FIX protein, most preferably the FIX transgene encodes for the Padua mutant of the human FIX protein. Accordingly, in a particularly preferred embodiment of the present invention, the transgene has SEQ ID NO: 11 (i.e. codon-optimized transgene encoding for the Padua mutant of the human FIX protein).

The term “coagulation factor VIII” has the meaning as known in the art. Synonyms of coagulation factor VIII are “FVIII” or “anti-hemophilic factor” or “AHF” and can be used interchangeably herein. The term “coagulation factor VIII” encompasses, for example, the human protein having the amino acid sequence as defined in Uniprot accession number P00451.

In embodiments, said FVIII is a FVIII wherein the B domain is deleted (i.e. B domain deleted FVIII, also referred to as BDD FVIII or FVIIIAB or FVIIIdeltaB herein). The term “B domain deleted FVIII” encompasses for example, but without limitation, FVIII mutants wherein whole or a part of the B domain is deleted and FVIII mutants wherein the B domain is replaced by a linker. Non-limiting examples of B domain deleted FVIII are described in Ward et al. (2011) and WO 2011/005968, which are specifically incorporated by reference in their entirety herein.

In preferred embodiments, said FVIII is B domain deleted FVIII wherein the B domain is replaced by a linker having the following sequence: SFSQNPPVLTRHQR (SEQ ID NO: 15) (i.e. SQ FVIII as defined in Ward et al. (2011)). In particularly preferred embodiments, said transgene encoding FVIII has SEQ ID NO: 16 (i.e. codon-optimized transgene encoding B domain deleted human FVIII, also referred to herein as (h)FVIIIcopt or co(h)FVIIIdeltaB or co(h)FVIIIdeltaB transgene), as disclosed also in WO 2011/005968, hereby incorporated by reference in its entirety.

In particular embodiments, said transgene in the nucleic acid molecules, expression cassettes and vectors described herein encodes for coagulation factor IX (FIX), preferably wherein said coagulation factor FIX contains a hyper-activating mutation, more preferably wherein said hyper-activating mutation corresponds to an R338L amino acid substitution, even more preferably wherein said transgene encodes for coagulation factor IX having a nucleic acid sequence defined by SEQ ID NO: 11.

In particular embodiments, said transgene in the nucleic acid molecules, expression cassettes and vectors described herein encodes for coagulation factor VIII (FVIII), preferably wherein said transgene is codon-optimized coagulation factor FVIII, or wherein said coagulation factor VIII has a deletion of the B domain, preferably wherein said B domain of said FVIII is replaced by a linker defined by SEQ ID NO: 15, more preferably wherein said transgene encodes for coagulation factor VIII having a nucleic acid sequence defined by SEQ ID NO: 16.

The term “coagulation factor VII” as used herein has the meaning as known in the art. Synonyms of coagulation factor VII are “FVII” and can be used interchangeably herein. The term “coagulation factor VII” encompasses, for example, the human protein having the amino acid sequence as defined in Uniprot accession number P08709. Coagulation FVII is typically convered into its active form “FVIIa” by proteolysis of the single peptide bond between amino acid residue at position 152 (arginine) and amino acid residue at position 163 (isoleucine), for example of the amino acid sequence as defined by SEQ ID NO: 30, leading to the formation of two polypeptide chains, a N-terminal light chain, for example of the amino acid sequence as defined by SEQ ID NO: 31, and a C-terminal heavy chain, for example of the amino acid sequence as defined by SEQ ID NO: 32, which are held together by one disulfide bridge. It was previously shown that fusing the coagulation factor VII protein or coagulation factor VIIa protein with albumin significantly prolongs the half-live of the FVII or FVIIaprotein (Schulte, 2008; Négrier, 2016; Herzog et al., 2014; Zollner et al., 2014; Metzner et al, 2013; Golor et al., 2013).

In particular embodiments, said transgene in the nucleic acid molecules, expression cassettes and vectors described herein encodes for coagulation factor VII, preferably wherein said transgene encoding FVII is codon-optimized. The term “coagulation factor VII” as used herein refers to a polypeptide or protein comprising the light chain, for example as defined by SEQ ID NO: 31, and the heavy chain of coagulation factor VII, for example as defined by SEQ ID NO: 32. Upon activation said light chain and heavy chain are coupled to each other by a disulfide bridge (i.e. resulting in “activated coagulation factor VII” or “FVIIa”). Accordingly, in particular embodiments, said transgene in the nucleic acid molecules, expression cassettes and vectors described herein encodes for the light chain and the heavy chain of coagulation factor VII, preferably wherein the light chain and the heavy chain of coagulation factor VII are separated from each other by one or more cleavable polypeptide or peptide linkers.

In particular embodiments, the one or more cleavable polypeptide or peptide linkers comprise at least amino acid sequence RKRRKR (SEQ ID NO: 33), RKR or PRPSRKRR (SEQ ID NO: 35), preferably RKRRKR (SEQ ID NO: 33), as previously described by Margaritis et al., 2004.

In preferred embodiments, said transgene in the nucleic acid molecules, expression cassettes and vectors described herein encodes for the light chain of factor VII as defined by SEQ ID NO: 31, the heavy chain as defined by SEQ ID NO: 32, and one or more cleavable polypeptide or peptide linkers comprising the sequence as defined by SEQ ID NO: 33. In more preferred embodiments, said transgene in the nucleic acid molecules, expression cassettes and vectors described herein encodes for the amino acid sequence as defined by SEQ ID NO: 34.

In particular embodiments, said transgene in the nucleic acid molecules, expression cassettes and vectors described herein is located at the 5′ end (i.e. upstream) of said sequence defined by SEQ ID NO: 14 or said sequence having at least 80% sequence identity to said sequence.

In particular embodiments, the protein or polypeptide encoded by the transgene and the human albumin encoded by the sequence defined by SEQ ID NO: 14 or said sequence having at least 80% sequence identity to said sequence) may be physically or spatially separated by, for example, an in-frame polypeptide or peptide linker.

Accordingly, in particular embodiments, said transgene in the nucleic acid molecules, expression cassettes and vectors described herein is separated from said sequence defined by SEQ ID NO: 14 or said sequence having at least 80% sequence identity to said sequence by a nucleic acid sequence encoding one or more polypeptide or peptide linkers.

As used herein, the term “linker” refers to a connecting element that serves to link other elements. Preferably, the linkage(s) between the transgene and the sequence defined by SEQ ID NO: 14 or said sequence having at least 80% sequence identity to said sequence, preferably wherein said transgene is a codon optimized transgene may be hydrolytically stable linkage(s), i.e., substantially stable in water at useful pH values, including in particular under physiological conditions.

In particular embodiments, the linker is a peptide linker of one or more amino acids. More particularly, the peptide linker may be 1 to 50 amino acids long or 2 to 50 amino acids long or 1 to 45 amino acids long or 2 to 45 amino acids long, preferably 1 to 40 amino acids long or 2 to 40 amino acids long or 1 to 35 amino acids long or 2 to 35 amino acids long, more preferably 1 to 30 amino acids long or 2 to 30 amino acids long. Further preferably, the linker may be 5 to 25 amino acids long or 5 to 20 amino acids long. Particularly preferably, the linker may be 5 to 15 amino acids long or 7 to 15 amino acids long. Hence, in certain embodiments, the linker may be 1, 2, 3 or 4 amino acids long. In other embodiments, the linker may be 5, 6, 7, 8 or 9 amino acids long. In further embodiments, the linker may be 10, 11, 12, 13 or 14 amino acids long. In still other embodiments, the linker may be 15, 16, 17, 18 or 19 amino acids long. In further embodiments, the linker may be 20, 21, 22, 23, 24 or 25 amino acids long.

The nature of amino acids constituting the linker is not of particular relevance as long as the biological activity of the polypeptide segments linked thereby is not substantially impaired and the linker provides for the intended spatial separation of the protein or polypeptide encoded by transgene and human albumin encoded by the sequence defined by SEQ ID NO: 14 or said sequence having at least 80% sequence identity to said sequence, preferably wherein said transgene is a codon optimized transgene. Preferred linkers are substantially non-immunogenic.

In certain preferred embodiments, the peptide linker may comprise, consist essentially of or consist of amino acids selected from the group consisting of Glycine (G), Serine (S), Alanine (A), Threonine (T), and combinations thereof. In even more preferred embodiments, the linker may comprise, consist essentially of or consist of amino acids selected from the group consisting of Glycine, Serine, and combinations thereof. Such linkers provide for particularly good flexibility. In certain embodiments, the linker may consist of only Glycine residues. In certain embodiments, the linker may consist of only Serine residues. For example, the polypeptide or peptide linker comprises, consists essentially of or consists of the amino acid sequence SSGGSGGSGGSGGSGGSGGSGGSGS (SEQ ID NO: 17) or a fragment thereof.

In particular embodiments, if the transgene encodes for the light chain and the heavy chain of coagulation factor VII (or factor FVIIa) as described elsewhere herein, the linker between albumin and the light and heavy chain of coagulation factor VII is preferably the linker as defined by SEQ ID NO: 17.

In particular embodiments, the polypeptide or peptide linker is a cleavable linker.

The term “cleavable” (sometimes also referred to as “biodegradable”), as used herein, refers to the capability of being split or divided, more particularly, dividing a complex molecule into simpler molecules. Proteolysis is the breakdown of proteins into smaller polypeptides or amino acids. Proteolysis is typically catalyzed by cellular enzymes called proteases. Low pH or high temperatures can also cause proteolysis without the need of enzymes. In vivo degradation of the fusion protein according to present invention, results in the release of the protein or polypeptide encoded by the transgene, which can subsequently induce its biological effect in a region of interest.

In particular embodiments, the polypeptide or peptide linker is cleavable by one or more proteases that are specific to the activation of a specific protein encoded by the transgene, preferably wherein the protein is a secretable therapeutic protein as described elsewhere herein.

In particular embodiments, e.g. if the transgene is FIX, the polypeptide or peptide linker is cleavable by one or more proteases that also activate wild-type FIX. In particular more embodiments, if the transgene is FIX, the polypeptide or peptide linker is derived from the cleavage site composed of the C-terminus of the FIX light chain and the N-terminus of the FIX activation peptide, allowing removing of human albumin in parallel to activation of FIX to increase the specific activity as described in Metzner et al., 2009. In more particular embodiments, if the transgene is FIX, the polypeptide or peptide linker comprises, consists essentially of or consists of an amino acid sequence SVSQTSKLTRAETVFPDVDGS (SEQ ID NO: 18), or a fragment thereof. The polypeptide or peptide linker consisting of an amino acid sequence as defined in SEQ ID NO: 18 may be encoded by a sequence as defined in SEQ ID NO: 29.

In particular embodiments of the nucleic acid molecule, said transgene is separated from said sequence defined by SEQ ID NO: 14 or said sequence having at least 80% sequence identity to said sequence by a sequence as defined in SEQ ID NO: 29 or a sequence having at least 85%, preferably at least 90%, more preferably at least 95%, such as 96%, 97%, 98% or 99%, identity to said sequence, or a functional fragment thereof.

A further aspect provides the fusion protein encoded by the nucleic acid molecule as taught herein. The fusion protein as taught herein typically comprises albumin and a transgene (or a transgene and coAlb), optionally coupled by one or more polypeptide or peptide linkers.

A further aspect provides nucleic acid expression cassettes for enhancing gene expression of a trangsgene and/or for increasing the levels and/or activity of a protein or polypeptide encoded by a transgene. More particularly, provided herein is a nucleic acid expression cassette comprising the nucleic acid molecule as taught herein, operably linked to a promoter.

As used herein, the term “nucleic acid expression cassette” refers to a nucleic acid molecule that includes one or more transcriptional control elements (such as, but not limited to promoters, enhancers and/or regulatory elements, polyadenylation sequences, and introns) that direct (trans)gene expression in one or more desired cell types, tissues or organs. Typically, the nucleic acid expression cassettes described herein will contain the nucleic acid molecule as taught herein.

The term “operably linked” as used herein refers to the arrangement of various nucleic acid molecule elements relative to each such that the elements are functionally connected and are able to interact with each other. Such elements may include, without limitation, a promoter, an enhancer and/or a regulatory element, a polyadenylation sequence, one or more introns and/or exons, and a coding sequence of a gene of interest to be expressed (i.e., the transgene) or a coding sequence of a fusion gene of interest to be expressed (e.g. the transgene fused to the sequence defined by SEQ ID NO: 14 or said sequence having at least 80% sequence identity to said sequence). The nucleic acid sequence elements, when properly oriented or operably linked, act together to modulate the activity of one another, and ultimately may affect the level of expression of the transgene. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements.

As used in the application, the term “promoter” refers to nucleic acid sequences that regulate, either directly or indirectly, the transcription of corresponding nucleic acid coding sequences to which they are operably linked (e.g. a transgene or endogenous gene). A promoter may function alone to regulate transcription or may act in concert with one or more other regulatory sequences (e.g. enhancers or silencers). In the context of the present application, a promoter is typically operably linked to regulatory elements to regulate transcription of a transgene and/or fusion gene. The promoter may be homologous (i.e. from the same species as the animal, in particular mammal, to be transfected with the nucleic acid expression cassette) or heterologous (i.e. from a source other than the species of the animal, in particular mammal, to be transfected with the expression cassette). As such, the source of the promoter may be any virus, any unicellular prokaryotic or eukaryotic organism, any vertebrate or invertebrate organism, or any plant, or may even be a synthetic promoter (i.e. having a non-naturally occurring sequence), provided that the promoter is functional in combination with the regulatory elements described herein. In preferred embodiments, the promoter is a mammalian promoter, in particular a murine or human promoter. Non-limiting examples of promoters include retroviral LTR promoter, particularly Rous sarcoma virus or Mouse Murine Leukemia Virus LTR, the cytomegalovirus (CMV) promoter, the SV40 promoter, the dihydrofolate reductase promoter, the β-actin promoter, the phosphoglycerol kinase (PGK) promoter, and the EF1α promoter.

The promoter may be an inducible or constitutive promoter.

In particular embodiments, the promoter contained in the nucleic acid expression cassettes and vectors disclosed herein is a tissue-specific promoter. Tissue-specific promoters are active in a specific type of cells or tissue, such as liver, B cells, T cells, hematopoietic cells, monocytic cells, leukocytes, macrophages, muscle, pancreatic acinar or beta cells, endothelial cells, astrocytes, neurons or lung.

In particular embodiments, the promoter contained in the nucleic acid expression cassettes and vectors disclosed herein is a liver-specific promoter, preferably a liver-specific promotor as described elsewhere herein. This is to increase liver specificity and/or avoid leakage of expression in other tissues.

The term “liver-specific promoter” encompasses any promoter that confers liver-specific expression to a (trans)gene. Non-limiting examples of liver-specific promoters are provided on the Liver Specific Gene Promoter Database (LSPD, http://rulai.cshl.edu/LSPD/), and include, for example, the transthyretin (TTR) promoter or TTR-minimal promoter (TTRm), the alpha 1-antitrypsin (AAT) promoter, the albumin (ALB) promotor or minimal promoter, the apolipoprotein A1 (APOA1) promoter or minimal promoter, the complement factor B (CFB) promoter, the ketohexokinase (KHK) promoter, the hemopexin (HPX) promoter or minimal promoter, the nicotinamide N-methyltransferase (NNMT) promoter or minimal promoter, the (liver) carboxylesterase 1 (CES1) promoter or minimal promoter, the protein C (PROC) promoter or minimal promoter, the apolipoprotein C3 (APOC3) promoter or minimal promoter, the mannan-binding lectin serine protease 2 (MASP2) promoter or minimal promoter, the hepcidin antimicrobial peptide (HAMP) promoter or minimal promoter, and the serpin peptidase inhibitor, clade C (antithrombin), member 1 (SERPINC1) promoter or minimal promoter.

The term “liver-specific expression” as used in the application, refers to the preferential or predominant expression of a (trans)gene (as RNA and/or polypeptide) in the liver, in liver tissue or in liver cells, as compared to other (i.e. non-liver) tissues or cells. According to particular embodiments, at least 50%, more particularly at least 60%, at least 70%, at least 75%, at least 80%, at least 85%, at least 90%, at least 95%, at least 98%, at least 99% or 100% of the (trans)gene expression occurs within liver tissue or liver cells. According to a particular embodiment, liver-specific expression entails that there is no ‘leakage’ of expressed gene product to other organs or tissue than liver, such as lung, muscle, brain, kidney and/or spleen. The same applies mutatis mutandis for hepatocyte-specific expression and hepatoblast-specific expression, which may be considered as particular forms of liver-specific expression. Throughout the application, where liver-specific is mentioned in the context of expression, hepatocyte-specific expression and hepatoblast-specific expression are also explicitly envisaged.

As used herein, the term “liver cells” encompasses the cells predominantly populating the liver and encompasses mainly hepatocytes, oval cells, liver sinusoidal endothelial cells (LSEC) and cholangiocytes (epithelial cells forming the bile ducts).

The term “hepatocyte,” as used herein, refers to a cell that has been differentiated from a progenitor hepatoblast such that it is capable of expressing liver-specific phenotype under appropriate conditions. The term “hepatocyte” also refers to hepatocytes that are de-differentiated. The term includes cells in vivo and cells cultured ex vivo regardless of whether such cells are primary or passaged.

The term “hepatoblast” as used herein, refers to an embryonic cell in the mesoderm that differentiates to give rise to a hepatocyte, an oval cell, or a cholangiocyte. The term includes cells in vivo and cells cultured ex vivo regardless of whether such cells are primary or passaged.

In particularly preferred embodiments, the promoter is a mammalian liver-specific promoter, in particular a murine or human liver-specific promoter.

According to a further particular embodiment, the liver-specific promoter is from the transthyretin (TTR) gene or from the Alpha-1-antitrypsin (AAT) gene. According to yet a further particular embodiment, the TTR promoter is a minimal promoter (also referred to as TTRm or TTR min herein), most particularly the minimal TTR promoter as defined in SEQ ID NO: 19.

According to particular embodiments, the promoter in the nucleic acid expression cassettes and vectors disclosed herein is a minimal promoter.

A ‘minimal promoter’ as used herein is part of a full-size promoter still capable of driving expression, but lacking at least part of the sequence that contributes to regulating (e.g. tissue-specific) expression. This definition covers both promoters from which (tissue-specific) regulatory elements have been deleted-that are capable of driving expression of a gene but have lost their ability to express that gene in a tissue-specific fashion and promoters from which (tissue-specific) regulatory elements have been deleted that are capable of driving (possibly decreased) expression of a gene but have not necessarily lost their ability to express that gene in a tissue-specific fashion. Minimal promoters have been extensively documented in the art, a non-limiting list of minimal promoters is provided in the specification.

Other sequences may be incorporated in the nucleic acid expression cassette disclosed herein as well, typically to further increase or stabilize the expression of the transgene and/or fusion gene product (e.g. introns and/or polyadenylation sequences).

Any intron can be utilized in the expression cassettes described herein. The term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of the polyadenylation signal. In embodiments, the nucleic acid expression cassette disclosed herein further comprises an intron. Non-limiting examples of suitable introns are Minute Virus of Mice (MVM) intron, beta-globin intron (betaIVS-II), factor IX (FIX) intron A, Simian virus 40 (SV40) small-t intron, and beta-actin intron. Preferably, the intron is an MVM intron, more preferably the MVM mini-intron as defined by SEQ ID NO: 20. The cloning of the MVM intron into a nucleic acid expression cassette described herein was shown to result in unexpectedly high expression levels of the transgene operably linked thereto.

Accordingly, in particular embodiments, the nucleic acid expression cassette comprises a minute virus of mice (MVM) intron, preferably the MVM intron defined by SEQ ID NO: 20.

Any polyadenylation signal that directs the synthesis of a polyA tail is useful in the expression cassettes described herein, examples of those are well known to one of skill in the art. Exemplary polyadenylation signals include, but are not limited to, polyA sequences derived from the Simian virus 40 (SV40) late gene, the bovine growth hormone (BGH) polyadenylation signal, the minimal rabbit (3-globin (mRBG) gene, and the synthetic polyA (SPA) site as described in Levitt et al. (1989, Genes Dev 3:1019-1025) (SEQ ID NO: 21). Preferably, the polyadenylation signal is the bovine growth hormone (BGH) polyadenylation signal (SEQ ID NO: 22) or the Simian virus 40 (SV40) polyadenylation signal (SEQ ID NO: 23).

Accordingly, in particular embodiments, the nucleic acid expression cassette comprises a transcriptional termination signal, preferably a polyadenylation signal, more preferably a synthetic polyadenylation signal (SEQ ID NO: 21) or the Simian Virus 40 (SV40) polyadenylation signal (SEQ ID NO: 23).

Typically, the nucleic acid expression cassette according to the invention comprises a promotor, an enhancer, a nucleic acid molecule as taught herein (e.g. a fusion gene comprising a transgene and a sequence defined by SEQ ID NO: 14 or a sequence having at least 80% sequence identity to said sequence) as taught herein, and a transcription terminator.

In particular embodiments, the nucleic acid expression cassette as taught herein comprises at least one (such as one, two, three, four, five or six, preferably three, (tandem) repeats) nucleic acid regulatory element operably linked to the promoter and the transgene fused to said sequence defined by SEQ ID NO: 14 or said sequence having at least 80% sequence identity to said sequence.

In particular embodiments, the nucleic acid expression cassette as taught herein comprises at least one (such as one, two, three, four, five or six, preferably three, (tandem) repeats) tissue-specific nucleic acid regulatory element operably linked to the promoter and the transgene fused to said sequence defined by SEQ ID NO: 14 or said sequence having at least 80% sequence identity to said sequence, preferably wherein the at least one tissue-specific nucleic acid regulatory element is a liver-specific nucleic acid regulatory element operably linked to the promoter and the transgene.

A “regulatory element” as used herein refers to transcriptional control elements, in particular non-coding cis-acting transcriptional control elements, capable of regulating and/or controlling transcription of a gene, in particular tissue-specific transcription of a gene. Regulatory elements comprise at least one transcription factor binding site (TFBS), more in particular at least one binding site for a tissue-specific transcription factor, suc as at least one binding site for a liver-specific transcription factor. Typically, regulatory elements as used herein increase or enhance promoter-driven gene expression when compared to the transcription of the gene from the promoter alone, without the regulatory elements. Thus, regulatory elements particularly comprise enhancer sequences, although it is to be understood that the regulatory elements enhancing transcription are not limited to typical far upstream enhancer sequences, but may occur at any distance of the gene they regulate. Indeed, it is known in the art that sequences regulating transcription may be situated either upstream (e.g. in the promoter region) or downstream (e.g. in the 3′UTR) of the gene they regulate in vivo, and may be located in the immediate vicinity of the gene or further away. Of note, although regulatory elements as disclosed herein typically are naturally occurring sequences, combinations of (parts of) such regulatory elements or several copies of a regulatory element, i.e. non-naturally occurring sequences, are themselves also envisaged as regulatory element. Regulatory elements as used herein may be part of a larger sequence involved in transcriptional control, e.g. part of a promoter sequence. However, regulatory elements alone are typically not sufficient to initiate transcription, but require a promoter to this end.

The one or more regulatory elements contained in the nucleic acid expression cassettes and vectors disclosed herein are preferably cell or tissue-specific, such as specific for liver, B cells, T cells, hematopoietic cells, monocytic cells, leukocytes, macrophages, muscle (e.g., regulatory elements as disclosed in WO 2015/110449, which is hereby incorporated by reference in its entirety), diaphragm (e.g., regulatory elements as disclosed in WO 2018/178067, which is hereby incorporated by reference in its entirety), pancreatic acinar or beta cells, endothelial cells (e.g., regulatory elements as disclosed in WO 2017/109039, which is hereby incorporated by reference in its entirety), astrocytes, neurons or lung.

In particular embodiments, the one or more regulatory elements contained in the nucleic acid expression cassettes and vectors disclosed herein are liver-specific. Non-limiting examples of liver-specific regulatory elements are disclosed in WO 2009/130208 and or WO 2016/146757, which are specifically incorporated by reference herein. Another example of a liver-specific regulatory element is a regulatory element derived from the transthyretin (TTR) gene, such as the regulatory element defined by SEQ ID NO: 24, also referred to herein as “TTRe” or “TTREnh” (Wu et al., 2008). ‘Liver-specific expression’, as used in the application, refers to the preferential or predominant expression of a (trans)gene (as RNA and/or polypeptide) in the liver as compared to other tissues. According to particular embodiments, at least 50% of the (trans)gene expression occurs within the liver. According to more particular embodiments, at least 60%, at least 70%, at least 75%, at least 80%, at least 85%, at least 90%, at least 95%, at least 97%, at least 99% or 100% of the (trans)gene expression occurs within the liver. According to a particular embodiment, liver-specific expression entails that there is no ‘leakage’ of expressed gene product to other organs, such as spleen, muscle, heart and/or lung. The same applies mutatis mutandis for hepatocyte-specific expression, which may be considered as a particular form of liver-specific expression. Throughout the application, where liver-specific is mentioned in the context of expression, hepatocyte-specific expression is also explicitly envisaged. Similarly, where tissue-specific expression is used in the application, cell-type specific expression of the cell type(s) predominantly making up the tissue is also envisaged.

Preferably, the one or more regulatory element in the nucleic acid expression cassettes and vectors disclosed herein is fully functional while being only of limited length. This allows its use in vectors or nucleic acid expression cassettes without unduly restricting their payload capacity. Accordingly, in embodiments, the one or more regulatory element in the expression cassettes and vectors disclosed herein is a nucleic acid of 1000 nucleotides or less, 800 nucleotides or less, or 600 nucleotides or less, preferably 400 nucleotides or less, such as 300 nucleotides or less, 200 nucleotides or less, 150 nucleotides or less, or 100 nucleotides or less (i.e. the nucleic acid regulatory element has a maximal length of 1000 nucleotides, 800 nucleotides, 600 nucleotides, 400 nucleotides, 300 nucleotides, 200 nucleotides, 150 nucleotides, or 100 nucleotides). However, it is to be understood that the disclosed nucleic acid regulatory elements retain regulatory activity (i.e. with regard to specificity and/or activity of transcription) and thus they particularly have a minimum length of 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, or 70 nucleotides.

In preferred embodiments, the one or more regulatory element in the nucleic acid expression cassettes and vectors disclosed herein comprises a sequence from SERPINA1 regulatory elements, i.e. regulatory elements that control expression of the SERPINA1 gene in vivo. Said regulatory element preferably comprises, consists essentially of or consists of the sequence as defined in SEQ ID NO: 25, a sequence having at least 85%, preferably at least 90%, more preferably at least 95%, such as 96%, 97%, 98% or 99%, identity to said sequence, or a functional fragment thereof. Also preferably, said regulatory element has a maximal length of 150 nucleotides or less, preferably 100 nucleotides or less, and comprises, consists essentially of or consists of the sequence as defined in SEQ ID NO: 25, a sequence having at least 85%, preferably at least 90%, more preferably at least 95%, such as 96%, 97%, 98% or 99%, identity to said sequence, or a functional fragment thereof. The liver-specific nucleic acid regulatory element consisting of SEQ ID NO: 25 is herein referred to as “the Serpin enhancer”, “SerpEnh”, or “Serp”.

The term “functional fragment” as used in the application refers to fragments of the sequences disclosed herein that retain the capability of regulating liver-specific expression, i.e. they still confer tissue specificity and they are capable of regulating expression of a (trans)gene in the same way (although possibly not to the same extent) as the sequence from which they are derived. Fragments comprise at least 10 contiguous nucleotides from the sequence from which they are derived. In further particular embodiments, fragments comprise at least 15, at least 20, at least 25, at least 30, at least 35 or at least 40 contiguous nucleotides from the sequence from which they are derived. Also preferably, functional fragments may comprise at least 1, more preferably at least 2, at least 3, or at least 4, even more preferably at least 5, at least 10, or at least 15, of the transcription factor binding sites (TFBS) that are present in the sequence from which they are derived.

In particularly preferred embodiments, the nucleic acid expression cassettes and vectors disclosed herein comprise two or more, such as two, three, four, five or six, preferably three, (tandem) repeats of a liver-specific regulatory element comprising, consisting essentially of or consisting of SEQ ID NO: 25, or a sequence having at least 85%, preferably at least 90%, more preferably at least 95%, such as 96%, 97%, 98% or 99%, identity to said sequence, more preferably a liver-specific regulatory element of 150 nucleotides or less, preferably 100 nucleotides or less, more preferably 80 nucleotides or less, comprising, consisting essentially of or consisting of SEQ ID NO: 25, or a sequence having at least 85%, preferably at least 90%, more preferably at least 95%, such as 96%, 97%, 98% or 99%, identity to said sequence. A preferred nucleic acid regulatory element comprising three tandem repeats of SEQ ID NO: 25 is herein referred to as “3×Serp” and is defined by SEQ ID NO: 26.

In more particular embodiments, the nucleic acid expression cassette as taught herein comprises at least one liver-specific nucleic acid regulatory element wherein the at least one liver-specific nucleic acid regulatory element consists of the Serpin enhancer defined by SEQ ID NO: 25 or a sequence having at least 95% identity to said sequence. In even more particular embodiments, the nucleic acid expression cassette as taught herein comprises a triple repeat, preferably tandemly arranged, of the Serpin enhancer defined by SEQ ID NO: 25 or the sequence having at least 95% identity to said sequence.

When a regulatory element as described herein is operably linked to both a promoter and a transgene and/or fusion gene, the regulatory element can (1) confer a significant degree of liver specific expression in vivo (and/or in hepatocytes/hepatic cell lines in vitro) of the transgene and/or fusion gene, and/or (2) can increase the level of expression of the transgene and/or fusion gene in the liver (and/or in hepatocytes/hepatocyte cell lines in vitro).

In a typical embodiment of the present invention, a nucleic acid expression cassette is disclosed and comprises:
a liver-specific nucleic acid regulatory element, preferably a regulatory element comprising one or three tandem repeats of the Serpin enhancer (e.g. SEQ ID NO: 25),
a liver-specific promoter, preferably the TTRm promoter (e.g. defined by SEQ ID NO: 19),
an intron, preferably the MVM intron, e.g. as defined by SEQ ID NO: 20, and
a (trans)gene, preferably the FIX, more preferably the codon-optimized factor IX cDNA as defined by SEQ ID NO: 9, even more preferably the hFIXcoPadua as defined by SEQ ID NO: 11;
a nucleic acid sequence encoding one or more polypeptide or peptide linker, preferably encoding a peptide linker as defined in SEQ ID NO: 18,
a sequence defined by SEQ ID NO: 14 or a sequence having at least 80% sequence identity to said sequence, and
a transcription terminator, preferably a polyadenylation signal such as the Simian vacuolating virus 40 or Simian virus 40 (SV40) polyadenylation signal as defined by SEQ ID NO: 23 or the synthetic polyA site as defined by SEQ ID NO: 21.

As a non-limiting example, such a vector is defined by SEQ ID NO: 2, SEQ ID NO: 3, SEQ ID NO: 6 or SEQ ID NO: 8, preferably SEQ ID NO: 3, SEQ ID NO: 6 or SEQ ID NO: 8, more preferably SEQ ID NO: 6 or SEQ ID NO: 8, even more preferably SEQ ID NO: 8.

The expression cassettes disclosed herein may be used as such, or typically, they may be part of a nucleic acid vector. Accordingly, a further aspect relates to the use of a nucleic acid expression cassette as described herein in a vector, in particular a nucleic acid vector.

A further aspect provides a vector comprising the nucleic acid expression cassette as taught herein, preferably wherein said vector is a viral vector, more preferably wherein said vector is derived from an adeno-associated virus (AAV).

The term ‘vector’ as used in the application refers to nucleic acid molecules, as single-stranded or double-stranded DNA, which may have inserted into it another nucleic acid molecule (the insert nucleic acid molecule) such as, but not limited to, a cDNA molecule. The vector is used to transport the insert nucleic acid molecule into a suitable host cell. A vector may contain the necessary elements that permit transcribing the insert nucleic acid molecule, and, optionally, translating the transcript into a polypeptide. The insert nucleic acid molecule may be derived from the host cell, or may be derived from a different cell or organism. Once in the host cell, the vector can replicate independently of, or coincidental with, the host chromosomal DNA, and several copies of the vector and its inserted nucleic acid molecule may be generated.

The term “vector” may thus also be defined as a gene delivery vehicle that facilitates gene transfer into a target cell. This definition includes both non-viral and viral vectors. Non-viral vectors include but are not limited to cationic lipids, liposomes, nanoparticles, PEG, PEI, etc. Viral vectors are derived from viruses including but not limited to: retrovirus, lentivirus, adeno-associated virus, adenovirus, herpesvirus, hepatitis virus or the like. Alternatively, gene delivery systems can be used to combine viral and non-viral components, such as nanoparticles or virosomes (Yamada et al., 2003).

Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Preferred vectors are derived from adeno-associated virus, adenovirus, retroviruses and lentiviruses.

Retroviruses and lentiviruses are RNA viruses that have the ability to insert their genes into host cell chromosomes after infection. Retroviral and lentiviral vectors have been developed that lack the genes encoding viral proteins, but retain the ability to infect cells and insert their genes into the chromosomes of the target cell (Miller, 1990; Naldini et al., 1996, VandenDriessche et al., 1999). The difference between a lentiviral and a classical Moloney-murine leukemia-virus (MLV) based retroviral vector is that lentiviral vectors can transduce both dividing and non-dividing cells whereas MLV-based retroviral vectors can only transduce dividing cells.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and nondividing cells in many different tissues in vivo including airway epithelial cells, endothelial cells, hepatocytes and various tumors (Trapnell, 1993; Chuah et al., 2003). Another viral vector is derived from the herpes simplex virus, a large, double-stranded DNA virus. Recombinant forms of the vaccinia virus, another dsDNA virus, can accommodate large inserts and are generated by homologous recombination.

Adeno-associated virus (AAV) is a small ssDNA virus which infects humans and some other primate species, not known to cause disease and consequently causing only a very mild immune response. AAV can infect both dividing and non-dividing cells and may incorporate its genome into that of the host cell. These features make AAV a very attractive candidate for creating viral vectors for gene therapy, although the cloning capacity of the vector is relatively limited. Accordingly, in preferred embodiments of the invention, the vector used is derived from adeno-associated virus (i.e. AAV vector).

Different serotypes of AAVs have been isolated and characterized, such as, for example AAV serotype 2, AAV serotype 5, AAV serotype 8, and AAV serotype 9, and all AAV serotypes are contemplated herein. In particular, AAV vectors that comprise a FIX transgene as disclosed herein are preferably AAV serotype 9 vectors, and AAV vectors that comprise a FVIII transgene as disclosed herein are preferably AAV serotype 8 vectors. The AAV vector may also be a non-naturally occurring AAV vector, such as AAV vectors derived from naturally-occurring vectors comprising capsids which are modified in such a way that they affect tropism or neutralisation by anti-AAV antibodies.

The AAV vectors disclosed herein may be single-stranded (i.e. ssAAV vectors) or self-complementary (i.e. scAAV vectors). In particular, AAV vectors that comprise a FIX transgene as disclosed herein are preferably self-complementary, and AAV vectors that comprise a FVIII transgene as disclosed herein are preferably single-stranded. With the term “self-complementary AAV” is meant herein a recombinant AAV-derived vector wherein the coding region has been designed to form an intra-molecular double-stranded DNA template.

Gene therapy with adeno-associated viral vectors disclosed herein was shown to induce immune tolerance towards the transgene comprised in the vector.

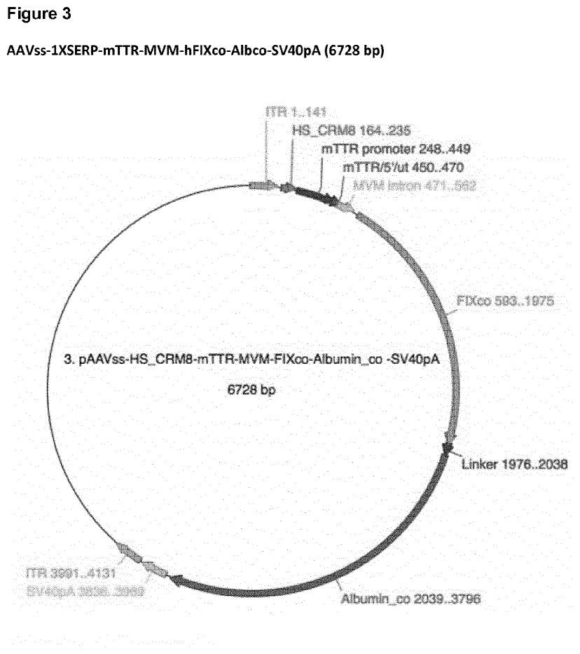

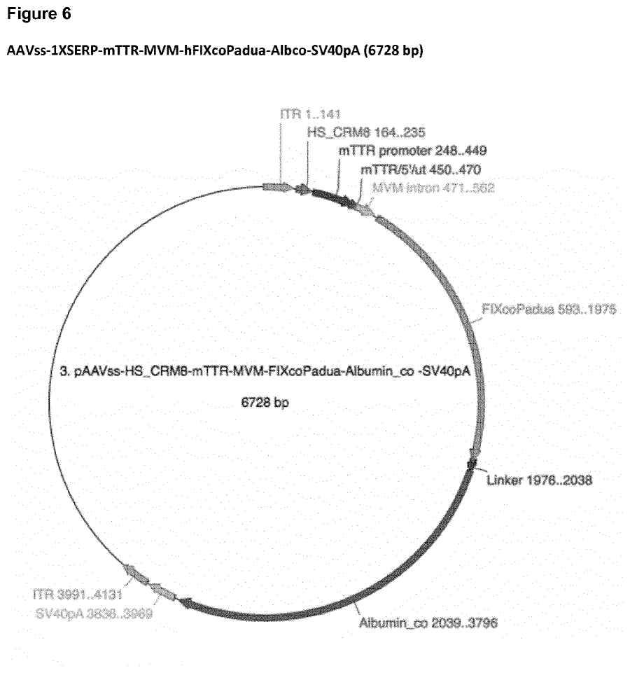

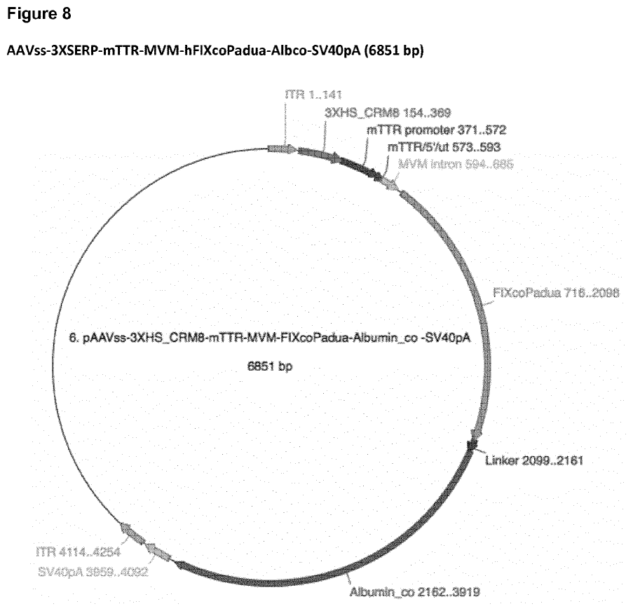

In embodiments, the vector according to the invention comprises the following elements (cfr. FIGS. 3, 6 and 8):
an Inverted Terminal Repeat sequence (ITR), optionally mutated,
a liver-specific regulatory element, preferably a regulatory element comprising one or three tandem repeats of the Serpin enhancer (“Serp” or “SerpEnh”) (e.g. a regulatory element comprising the nucleic acid fragment defined by SEQ ID NO: 25),
a promoter, preferably the minimal TTR promoter (TTRm) defined by SEQ ID NO: 19,
an intron, preferably the MVM intron defined by SEQ ID NO: 20,
a (trans)gene, preferably codon-optimized factor IX cDNA defined by SEQ ID NO: 9, even more preferably codon-optimized factor IX Padua cDNA defined by SEQ ID NO: 11,
a nucleic acid sequence encoding one or more polypeptide or peptide linker, preferably encoding a peptide linker as defined in SEQ ID NO: 18,
a sequence defined by SEQ ID NO: 14 or a sequence having at least 80% sequence identity to said sequence,
a transcription terminator, preferably a polyadenylation signal such as the Simian vacuolating virus 40 or Simian virus 40 (SV40) polyadenylation signal defined by SEQ ID NO: 23 or the synthetic polyA site as defined by SEQ ID NO: 21, and
an Inverted Terminal Repeat sequence (ITR).

The combination of said elements resulted in an unexpectedly high protein level and activity of codon optimized FIX or the codon optimized FIX with the Padua mutation in the liver of subjects, while this was not observed when vectors comprising the wild-type, non-codon optimised form of human albumin cDNA instead of the a sequence defined by SEQ ID NO: 14 or a sequence having at least 80% sequence identity to said sequence were used.

Preferably, the vector is an adeno-associated virus-derived vector, more preferably a self-complementary AAV vector, even more preferably a self-complementary AAV serotype 9 vector, such as the vector as defined by SEQ ID NO: 2, SEQ ID NO: 3, SEQ ID NO: 6 or SEQ ID NO: 8, SEQ ID NO: 3, SEQ ID NO: 6 or SEQ ID NO: 8, more preferably SEQ ID NO: 6 or SEQ ID NO: 8, even more preferably SEQ ID NO: 8.

In particular embodiments, said vector is a single-stranded AAV.

In specific embodiments the following plamids/vectors are provided:

(SEQ ID NO: 2)
pAAVss-1XSERP-mTTR-MVM-hFIXco-Albco-SV40pA,
(SEQ ID NO: 6)
pAAVss-1XSERP-mTTR-MVM-hFIXcoPadua-Albco-SV40pA,
(SEQ ID NO: 3)
pAAVss-3XSERP-mTTR-MVM-hFIXco-Albco-SV40pA,
and
(SEQ ID NO: 8)
pAAVss-3XSERP-mTTR-MVM-hFIXcoPadua-Albco-SV40pA.

In particular embodiments, the vector as taught herein has SEQ ID NO: 2, SEQ ID NO: 3, SEQ ID NO: 6 or SEQ ID NO: 8, preferably SEQ ID NO: 3, SEQ ID NO: 6 or SEQ ID NO: 8, more preferably SEQ ID NO: 6 or SEQ ID NO: 8, even more preferably SEQ ID NO: 8. In other embodiments, the vector is a non-viral vector, such as a transposon-based vector. Preferably, said transposon-based vectors are derived from Sleeping Beauty (SB) or PiggyBac (PB). A preferred SB transposon has been described in Ivies et al. (1997) and its hyperactive versions, including SB100X, as described in Mates et al. (2009). PiggyBac-based transposons are safe vectors in that they do no enhance the tumorigenic risk. Furthermore, liver-directed gene therapy with these vectors was shown to induce immune tolerance towards the transgene, in particular the hFIX or hFVIII transgene, comprised in the vector.

The transposon-based vectors are preferably administered in combination with a vector encoding a transposase for gene therapy. For example, the PiggyBac-derived transposon-based vector can be administered with wild-type PiggyBac transposase (Pbase) or mouse codon-optimized PiggyBac transposase (mPBase) Preferably, said transposases are hyperactive transposases, such as, for example, hyperactive PB (hyPB) transposase containing seven amino acid substitutions (I30V, S103P, G165S, M282V, S509G, N538K, N570S) as described in Yusa et al. (2011), which is specifically incorporated by reference herein.

Transposon/transposase constructs can be delivered by hydrodynamic injection or using non-viral nanoparticles to transfect cells, such as hepatocytes.

A further aspect provides a pharmaceutical composition or pharmaceutical preparation comprising the nucleic acid molecule, the nucleic acid expression cassete or the vector as taught herein, and a pharmaceutically acceptable carrier, i.e., one or more pharmaceutically acceptable carrier substances and/or additives, e.g., buffers, carriers, excipients, stabilisers, etc. The pharmaceutical composition may be provided in the form of a kit.

The term “pharmaceutically acceptable” as used herein is consistent with the art and means compatible with the other ingredients of the pharmaceutical composition and not deleterious to the recipient thereof. The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine. The pharmaceutical composition according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The pharmaceutical composition can be prepared in a manner known per se to one of skill in the art. For this purpose, the nucleic acid expression cassette or the expression vector as defined herein, one or more solid or liquid pharmaceutically acceptable excipients and, if desired, in combination with other pharmaceutical active compounds, are brought into a suitable administration form or dosage form which can then be used as a pharmaceutical in human medicine or veterinary medicine.

According to another aspect, a pharmaceutical composition is provided comprising a nucleic acid molecule comprising a transgene encoding a therapeutic protein fused to the sequence defined by SEQ ID NO: 14 or said sequence having at least 80% sequence identity to said sequence as taught herein or a nucleic acid expression cassette comprising such a nucleic acid molecule as taught herein, and a pharmaceutically acceptable carrier.

According to another embodiment, the pharmaceutical composition comprises a vector comprising the nucleic acid expression cassette comprising a nucleic acid molecule comprising a transgene encoding a therapeutic protein fused to the sequence defined by SEQ ID NO: 14 or said sequence having at least 80% sequence identity to said sequence as taught herein, and a pharmaceutically acceptable carrier. According to further particular embodiments, the transgene encodes factor IX and the pharmaceutical composition is for treating hemophilia B or the transgene encodes factor VIII and the pharmaceutical composition is for treating hemophilia A.

A further aspect provides the use of the nucleic acid molecules, the nucleic acid expression cassettes, the vectors, the pharmaceutical compositions as taught herein for enhancing gene expression of a transgene and/or for increasing the levels and/or activity of a protein or polypeptide encoded by a transgene, wherein said use is an in vitro, ex vivo or in vivo use, preferably wherein said use is an in vitro use.

In particular embodiments, the level of the fusion protein encoded by the nucleic acid molecule comprising a transgene fused to a sequence defined by SEQ ID NO: 14 or said sequence having at least 80% sequence identity to said sequence as taught herein in vitro or in vivo (e.g. circulating proteins or polypeptides) is from about 2-fold to about 5-fold, more—when compared to the level of the protein or polypeptide encoded by the same transgene as present in the nucleic acid molecule encoding the fusion protein, in absence of fusion to albumin, in vitro or in vivo. The level of a protein or polypeptide may be determined by any art-recognized means, such as by antibody-based assays, e.g. a Western Blot or an ELISA assay, for instance to evaluate whether therapeutic expression of the gene product is achieved. Expression of the gene product may also be measured in a bioassay that detects an enzymatic or biological activity of the gene product as described elsewhere herein.

In particular embodiments, the activity of the fusion protein encoded by the nucleic acid molecule comprising a transgene fused to a sequence defined by SEQ ID NO: 14 or said sequence having at least 80% sequence identity to said sequence as taught herein is from about 1.5 to about 4-fold more—when compared to the activity of the protein or polypeptide encoded by the same transgene as present in the nucleic acid molecule encoding the fusion protein, in absence of fusion to albumin. Reference to the “activity” of a polypeptide or protein may generally encompass any one or more aspects of the biological activity of the polypeptide or protein, such as without limitation any one or more aspects of its biochemical activity, enzymatic activity, signaling activity, interaction activity, ligand activity, and/or structural activity, e.g., within a cell, tissue, organ or an organism. The activity of a protein or polypeptide may be determined by any methods known in the art and is dependent on the type of protein or polypeptide and the type of activity. For example, if the protein or polypeptide is FIX, the activity may be determined using a chromogenic assay (HYPHEN BioMed, Andresy, France).

In particular embodiments, the nucleic acid molecules, the nucleic acid expression cassettes, the vectors and the pharmaceutical compositions as taught herein increase the half-life (which may be reflected by a higher steady-state protein level) in vitro as well as the circulatory half-life (which may be reflected by a higher steady-state protein level) in vivo of the fusion protein encoded by a nucleic acid molecule comprising a transgene fused to the sequence defined by SEQ ID NO: 14 or said sequence having at least 80% sequence identity to said sequence as taught herein, and therefore as well as the half-life of the protein or polypeptide encoded by the transgene. More particularly, the steady-state level of the fusion proteins as taught herein (as well as the steady-state level of the protein or polypeptide encoded by the transgene) is about 1.5-fold to 5-fold more—when compared to the steady-state protein level of the protein or polypeptide encoded by the same transgene as in the fusion protein but not being fused to albumin. The half-life of a protein or polypeptide may be determined by any methods known in the art, for example by pharmacokinetic studies.

Genetic fusion of albumin to a protein or polypeptide of interest, such as a therapeutic protein, improves the pharmacokinetic properties of said protein or polypeptide of interest, more particularly, it extends the half-life thereof. The fusion proteins as taught herein may be considered a long-acting fusion protein, compared to proteins or polypeptides not fused to human albumin.

Furthermore, the expression cassettes and vectors described herein direct the expression of a therapeutic amount of the fusion gene product for an extended period. Typically, therapeutic expression is envisaged to last at least 20 days, at least 50 days, at least 100 days, at least 200 days, at least 300 days, at least 1 year, at least 2 years, at least 3 years, at least 4 years, at least 5 years, at least 6 years, at least 7 years, at least 8 years, at least 9 years and in some instances ten years or more. In a further aspect, the nucleic acid molecules, the nucleic acid expression cassettes and the vectors described herein can be used in gene therapy.

Gene therapy protocols, intended to achieve therapeutic gene product expression in target cells, in vitro, but also particularly in vivo, have been extensively described in the art. These include, but are not limited to, intramuscular injection of plasmid DNA (naked or in liposomes), interstitial injection, instillation in airways, application to endothelium, intra-hepatic parenchyme, and intravenous or intra-arterial administration (e.g. intra-hepatic artery, intra-hepatic vein). Various devices have been developed for enhancing the availability of DNA to the target cell. A simple approach is to contact the target cell physically with catheters or implantable materials containing DNA. Another approach is to utilize needle-free, jet injection devices which project a column of liquid directly into the target tissue under high pressure. These delivery paradigms can also be used to deliver viral vectors. Another approach to targeted gene delivery is the use of molecular conjugates, which consist of protein or synthetic ligands to which a nucleic acid- or DNA-binding agent has been attached for the specific targeting of nucleic acids to cells (Cristiano et al., 1993).

According to particular embodiments, the use of the nucleic acid molecules, the nucleic acid expression cassettes and vectors as described herein is envisaged for gene therapy of a specific type of cells or tissue (e.g., liver (i.e. liver-directed gene therapy), muscle (i.e. muscle-directed gene therapy), endothelial cells (i.e. endothelium-specific gene therapy)), preferably of liver cells (i.e. liver-directed gene therapy). According to a further particular embodiment, the use of the nucleic acid molecules, expression cassettes or vectors is for gene therapy, in particular liver-directed gene therapy, in vivo. According to yet a further particular embodiment, the use is for a method of gene therapy, in particular liver-directed gene therapy, to treat hemophilia, in particular to treat hemophilia B or hemophilia A.

Gene transfer into mammalian hepatocytes has been performed using both ex vivo and in vivo procedures. The ex vivo approach requires harvesting of the liver cells, in vitro transduction with long-term expression vectors, and reintroduction of the transduced hepatocytes into the portal circulation (Kay et al., 1992; Chowdhury et al., 1991). In vivo targeting has been done by injecting DNA or viral vectors into the liver parenchyma, hepatic artery, or portal vein, as well as via transcriptional targeting (Kuriyama et al., 1991; Kistner et al., 1996). Recent methods also include intraportal delivery of naked DNA (Budker et al., 1996) and hydrodynamic tail vein transfection (Liu et al., 1999; Zhang et al., 1999).

According to a further aspect, methods for expressing a protein in cells, preferably liver cells, are provided, comprising the steps of
introducing in cells, preferably liver cells, the nucleic acid expression cassette or a vector as described herein, and
expressing the fusion gene product in the cells, preferably liver cells.

These methods may be performed both in vitro and in vivo.

A further aspect provides the use of the nucleic acid molecules, the nucleic acid expression cassettes, the vectors, the pharmaceutical compositions as taught herein for treating a disease or disorder, preferably by gene therapy.

Methods of gene therapy for a subject in need thereof are also provided, comprising the steps of introducing in an organ, preferably the liver, of the subject a nucleic acid expression cassette comprising a nucleic acid molecule comprising a transgene fused to the sequence defined by SEQ ID NO: 14 or said sequence having at least 80% sequence identity to said sequence wherein said transgene encodes a therapeutic protein, and expressing a therapeutic amount of the therapeutic protein in said organ, preferably the liver. According to a further embodiment, the method comprises the steps of introducing in an organ, preferably the liver, of the subject a vector comprising the nucleic acid expression cassette comprising a nucleic acid molecule comprising a transgene fused to the sequence defined by SEQ ID NO: 14 or said sequence having at least 80% sequence identity to said sequence wherein said transgene encodes a therapeutic protein, and expressing a therapeutic amount of the therapeutic protein in said organ, preferably the liver.

Exemplary diseases and disorders that may benefit from gene therapy using the nucleic acid molecules encoding human albumin, the nucleic acid expression cassettes, the vectors, or the pharmaceutical compositions as taught herein include liver diseases, liver-related diseases such as haemophilia (including hemophilia A and B), myotubular myopathy (MTM), Pompe disease, muscular dystrophy (e.g. Duchenne muscular dystrophy (DMD)/Becker muscular dystrophy (BMD)), myotonic dystrophy, Myotonic Muscular Dystrophy (DM), Miyoshi myopathy, Fukuyama type congenital, muscular dystrophy, dysferlinopathies neuromuscular disease, motor neuron diseases (MND), such as Charcot-Marie-Tooth disease (CMT), spinal muscular atrophy (SMA), and amyotrophic lateral sclerosis (ALS), Emery-Dreifuss muscular dystrophy, facioscapulohumeral muscular dystrophy (FSHD), congenital muscular dystrophies, congenital myopathies, limb girdle muscular dystrophy, metabolic myopathies, muscle inflammatory diseases, myasthenia, mitochondrial myopathies, anomalies of ionic channels, nuclear envelop diseases, cardiomyopathies, cardiac hypertrophy, heart failure, distal myopathies, cardiovascular diseases, von Willebrand disease, microvascular thrombosis, thrombotic thrombocytopenic purpura, peripheral vascular disease, coronary artery diseases, atherosclerotic diseases, stroke, heart disease, diabetes, insulin resistance, chronic kidney failure, tumor growth, metastasis, venous thrombosis, ischemia, tumour growth, tumour vascularisation, cancer and viral infectious diseases such as Ebola, Dengue fever, dengue hemorrhagic fever, autoimmune disease (e.g. Crohn's, multiple sclerosis and lysosomal storage diseases.

In particular embodiments, the disease or disorder is a liver-related disease or disorder.

The term “liver-related disease or disorder” as used herein refers to a disease or disorder associated with altered gene expression in the liver. Liver-related diseases or disorders include hepatic diseases sensu stricto as well as some hereditary disorders that do not directly lead to liver disease but manifest themselves primarily elsewhere in the body. Non-limiting examples of liver-related diseases or disorders include hemophilia (including haemophilia A and B), hepatitis, cancer, and cirrhosis, Polycystic liver disease (PLD), hemophilia A or B, familial hypercholesterolemia, lysosomal storage diseases, ornithine transcarbamylase deficiency and α-antitrypsin deficiency.

The choice of the transgene as well as the tissue-specific promoter and/or tissue-specific regulatory element(s) is typically linked to the disease(s) or disorder(s) which are intended to be treated using the vector or the pharmaceutical composition as taught herein. For example, if the disease or disorder which is intended to be treated is a liver-related disorder, such as hemophilia, the promoter and the regulatory elements used are preferably liver- or hepatocyte-specific and the transgene is preferably FIX, FVIII or FVII, more preferably FIX or FVIII.

According to a very specific embodiment, the therapeutic protein encoded by the transgene in the nucleic acid expression cassette or the vector is factor IX, and the method is a method for treating hemophilia B. By expressing factor IX in the liver via gene therapy, hemophilia B can be treated (Snyder et al., 1999). According to another very specific embodiment, the therapeutic protein encoded by the transgene in the nucleic acid expression cassette or the vector is factor VIII, and the method is a method for treating hemophilia A. According to another very specific embodiment, the therapeutic protein encoded by the transgene in the nucleic acid expression cassette or the vector is factor VII (or coagulation factor VIIa), and the method is a method for treating hemophilia A; hemophilia B or FVII deficiency.

Also provided herein is a method of treating a disease or disorder that may benefit from gene therapy as described elsewhere herein, preferably a liver-related disorder, more preferably hemophilia, in a subject in need of such a treatment, comprising administering a therapeutically effective amount of vector or the pharmaceutical composition as taught herein to the subject.

Except when noted differently, the terms “subject” or “patient” are used interchangeably and refer to animals, preferably vertebrates, more preferably mammals, and specifically includes human patients and non-human mammals, such as e.g. mice. Preferred patients or subjects are human subjects.

As used herein, the terms “treat” or “treatment” refer to both therapeutic treatment and prophylactic or preventative measures, wherein the object is to prevent or slow down (lessen) an undesired physiological change or disorder, such as the development or spread of proliferative disease, e.g., cancer. Beneficial or desired clinical results include, but are not limited to, alleviation of symptoms, diminishment of extent of disease, stabilised (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a phrase such as “a subject in need of treatment” includes subjects, such as mammalian subjects, that would benefit from treatment of a given condition, such as, hemophilia B or hemophilia A. Such subjects will typically include, without limitation, those that have been diagnosed with the condition, those prone to have or develop the said condition and/or those in whom the condition is to be prevented.

The term “therapeutically effective amount” refers to an amount of a compound or pharmaceutical composition effective to treat a given condition in a subject, i.e., to obtain a desired local or systemic effect and performance. The term thus refers to the quantity of compound or pharmaceutical composition that elicits the biological or medicinal response in a tissue, system, animal, or human that is being sought by a researcher, veterinarian, medical doctor or other clinician, which includes alleviation of the symptoms of the disease or disorder being treated. In particular, these terms refer to the quantity of compound or pharmaceutical composition according to the invention which is necessary to prevent, cure, ameliorate, or at least minimize the clinical impairment, symptoms, or complications associated with a given condition, such as hemophilia if therapeutic protein encoded by the transgene is factor IX or VIII, in either a single or multiple dose.

In a particular embodiment, if the therapeutic protein encoded by the transgene is factor IX (and the transgene is fused to a sequence defined by SEQ ID NO: 14 or a sequence having at least 80% sequence identity to said sequence), the term implies that levels of factor IX in plasma are equal to or higher than the therapeutic concentration of at least about 1% of physiological activity, i.e. 10 mU/ml (milli-units per milliliter) plasma, at least 5% of physiological activity or 50 mU/ml plasma, at least 10% of physiological activity or 100 mU/ml plasma, at least 15% of physiological activity or 150 mU/ml, at least 20% of physiological activity or 200 mU/ml plasma, at least 25% of physiological activity or 250 mU/ml, at least 30% of physiological activity or 300 mU/ml, at least 35% of physiological activity or 350 mU/ml, at least 40% of physiological activity or 400 mU/ml, at least 45% of physiological activity or 400 mU/ml, at least 45% of physiological activity or 450 mU/ml, at least 50% of physiological activity or 500 mU/ml, at least 65% of physiological activity or 650 mU/ml, at least 70% of physiological activity or 700 mU/ml, at least 75% of physiological activity or 750 mU/ml, at least 80% of physiological activity or 800 mU/ml, at least 85% of physiological activity or 850 mU/ml, at least 95% of physiological activity or 950 mU/ml, or at least 100% of physiological activity or 1000 mU/ml, in a subject can be obtained by transduction or transfection of the vector according to any one the embodiments described herein into a subject. Due to the very high efficiency of the nucleic acid expression cassettes and vectors of the present invention and/or the longer half-life of the fusion gene product obtained therefrom, this high therapeutic levels of factor IX in the subject can be obtained even by administering relatively low doses of vector.

In another particular embodiment, if the therapeutic protein encoded by the transgene is factor VIII (and the transgene is fused to a sequence defined by SEQ ID NO: 14 or a sequence having at least 80% sequence identity to said sequence), the term implies that through levels of factor VIII in plasma equal to or higher than the therapeutic concentration of 10 mU/ml (milli-units per milliliter) plasma, 50 mU/ml plasma, 100 mU/ml plasma, 150 mU/ml plasma, 200 mU/ml plasma, 250 mU/ml plasma, 300 mU/ml plasma, 350 mU/ml plasma, 400 mU/ml plasma, 450 mU/ml plasma, 500 mU/ml plasma, 550 mU/ml plasma, 600 mU/ml plasma, 650 mU/ml plasma, 750 mU/ml plasma, 800 mU/ml plasma, 850 mU/ml plasma, 900 mU/ml plasma, 950 mU/ml plasma, or higher can be obtained by transduction or transfection of any of the vectors disclosed herein into a subject. Due to the very high efficiency of the vectors and nucleic acid expression cassettes disclosed herein and/or the longer half-life of the fusion gene product (i.e. fusion protein) obtained therefrom, these high therapeutic levels of factor VIII in the subject can be obtained even by administering relatively low doses of vector.

In another particular embodiment, if the therapeutic protein encoded by the transgene is factor VII or coagulation factor VIIa (and the transgene is fused to a sequence defined by SEQ ID NO: 14 or a sequence having at least 80% sequence identity to said sequence), the term implies that trough levels of factor VII or factor VIIa in plasma equal to or higher than the therapeutic concentration of 10 U/ml (units per milliliter) plasma, 50 U/ml plasma, 100 U/ml plasma, or higher (or equal to or higher than the therapeutic concentrations as described in Abshire et al., 2004) can be obtained by transduction or transfection of any of the vectors disclosed herein into a subject. Due to the very high efficiency of the vectors and nucleic acid expression cassettes disclosed herein and/or the longer half-life of the fusion gene product (i.e. fusion protein) obtained therefrom, these high therapeutic levels of factor VII (or factor VIIa) in the subject can be obtained even by administering relatively low doses of vector.

A further aspect provides the vector or the pharmaceutical composition as taught herein for use as a medicament.

A further aspect provides the vector or the pharmaceutical composition as taught herein for use in the treatment of a disease or a disorder that may benefit from gene therapy as described elsewhere herein, preferably a liver-related disorder, more preferably hemophilia if the transgene encodes for FIX, FVIII or FVII.

Also provided herein is the use of the vector or the pharmaceutical composition as taught herein for the manufacture of a medicament for the treatment of a disease or a disorder that may benefit from gene therapy as described elsewhere herein, preferably a liver-related disorder, more preferably hemophilia, in a subject.

In particular embodiments, if the transgene encodes factor IX or factor VIII, the transduction of the vector according to any one of the embodiments defined herein into the subject can be done at a dose lower than 6×10¹³ vg/kg (viral genomes per kilogram) to obtain a therapeutic factor IX level of 100 mU/ml plasma or higher in a subject. For example, a level of factor IX of 300 mU/ml plasma or higher in a subject may be achieved at a dose lower than 5×10¹¹ vg/kg.

For hemophilia therapy, efficacy of the treatment can, for example, be measured by assessing the hemophilia-caused bleeding in the subject. In vitro tests such as, but not limited to the in vitro activated partial thromboplastin time assay (APPT), test factor IX chromogenic activity assays, blood clotting times, factor IX or human factor VIII-specific ELISAs are also available. Any other tests for assessing the efficacy of the treatment known in the art can of course be used.

The nucleic acid expression cassette, the vector or the pharmaceutical composition of the invention may be used alone or in combination with any of the known therapies for a given condition. For example, known hemophilia therapies include the administration of recombinant or purified clotting factors. The nucleic acid expression cassette, the vector or the pharmaceutical composition of the invention can thus be administered alone or in combination with one or more active compounds. The latter can be administered before, after or simultaneously with the administration of the said agent(s).

The use of the nucleic acid molecule, the nucleic acid expression cassette and the vector components as disclosed herein for the manufacture of these pharmaceutical compositions for use in treating a given condition, preferably a liver-related disease, more preferably hemophilia, even more preferably hemophilia B or hemophilia A, is also envisaged.

In an alternative example, the nucleic acid molecule, the expression cassettes and vectors disclosed herein may be used to express an immunological amount of a gene product (such as a polypeptide, in particular an immunogenic protein, or RNA) for vaccination purposes.

In embodiments, the pharmaceutical composition may be a vaccine. The vaccine may further comprise one or more adjuvants for enhancing the immune response. Suitable adjuvants include, for example, but without limitation, saponin, mineral gels such as aluminium hydroxide, surface active substances such as lysolecithin, pluronic polyols, polyanions, peptides, oil or hydrocarbon emulsions, bacilli Calmette-Guerin (BCG), Corynebacterium parvum, and the synthetic adjuvant QS-21. Optionally, the vaccine may further comprise one or more immunostimulatory molecules. Non-limiting examples of immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

In embodiments, the nucleic acid molecules, the nucleic acid expression cassettes, the vectors, or the pharmaceutical compositions described herein may be for use as a vaccine, more particularly for use as a prophylactic vaccine.

Also disclosed herein is the use of the nucleic acid molecules, the nucleic acid regulatory elements, the nucleic acid expression cassettes, the vectors, or the pharmaceutical compositions described herein for the manufacture of a vaccine, in particular for the manufacture of a prophylactic vaccine.

Also disclosed herein is a method of vaccination, in particular prophylactic vaccination, of a subject in need of said vaccination comprising:
introducing in the subject, in particular in liver of the subject, a nucleic acid expression cassette, a vector or a pharmaceutical composition as taught herein; wherein the nucleic acid expression cassette, the vector or the pharmaceutical composition comprises at least a transgene fused to a sequence defined by SEQ ID NO: 14 or a sequence having at least 80% sequence identity to said sequence operably linked to a promoter; and
expressing an immunologically effective amount of the fusion gene product (i.e. fusion protein) in the subject, in particular in liver of the subject.

An “immunologically effective amount” as used herein refers to the amount of (trans)gene product effective to enhance the immune response of a subject against a subsequent exposure to the immunogen encoded by the (trans)gene. Levels of induced immunity can be determined, e.g. by measuring amounts of neutralizing secretory and/or serum antibodies, e.g., by plaque neutralization, complement fixation, enzyme-linked immunosorbent, or microneutralization assay.

It is to be understood that although particular embodiments, specific constructions and configurations, as well as materials, have been discussed herein for methods and applications according to the present invention, various changes or modifications in form and detail may be made without departing from the scope and spirit of this invention.

The following examples are provided to better illustrate particular embodiments, and they should not be considered limiting the application. The application is limited only by the claims.

### EXAMPLES

### Example 1: Codon-Optimalisation of Human Albumin

Codon-optimized sequences were created by analysis of the known human albumin cDNA (SEQ ID NO: 28) and codon usage adapted to the codon bias of Homo sapiens using codon adaptation index performed by GeneArt (GeneArt AG) using their in-house proprietary software GeneOptimizer.

Negative cis-acting sites (such as splice donor and acceptor sites, internal TATA-boxes, chi-sites and ribosomal entry sites, RNA instability motifs, repeat sequences and RNA secondary structures etc.) which may negatively influence expression were elimitated wherever possible.

GC content was adjusted to prolong mRNA half life. More particularly, regions of very high (>80%) or very low (<30%) GC content were avoided where possible.

Codon usage resulted in a codon adaptation index (CAI) value of 0.96 (see Nucleic Acids Res. 1987 Feb. 11; 15(3):1281-95; The codon Adaptation Index—a measure of directional synonymous codon usage bias, and its potential applications.; Sharp P M, Li W H). The CAI describes how well the codons match the codon usage preference of the target organism, and is preferably >0.9.

### Example 2: hFIXco-Albco Fusions Result in a Robust Increase in Steady-State hFIX Levels and Activity

1. Study Design

The following vector constructs were generated by conventional cloning and synthetic gene assembly:

1)
(SEQ ID NO: 1)
AAVss-1XSERP-mTTR-MVM-hFIXco-SV40pA
2)
(SEQ ID NO: 2)
AAVss-1XSERP-mTTR-MVM-hFIXco-Albco-SV40pA
3)
(SEQ ID NO: 3)
AAVss-3XSERP-mTTR-MVM-hFIXco-Albco-SV40pA
4)
(SEQ ID NO: 4)
AAVss-1XSERP-mTTR-MVM-hFIXcoPadua-SV40pA
5)
(SEQ ID NO: 5)
AAVss-1XSERP-mTTR-MVM-hFIXcoPadua-Alb-SV40pA
6)
(SEQ ID NO: 6)
AAVss-1XSERP-mTTR-MVM-hFIXcoPadua-Albco-SV40pA
7)
(SEQ ID NO: 7)
AAVss-3XSERP-mTTR-MVM-hFIXcoPadua-Alb-SV40pA
8)
(SEQ ID NO: 8)
AAVss-3XSERP-mTTR-MVM-hFIXcoPadua-Albco-SV40pA

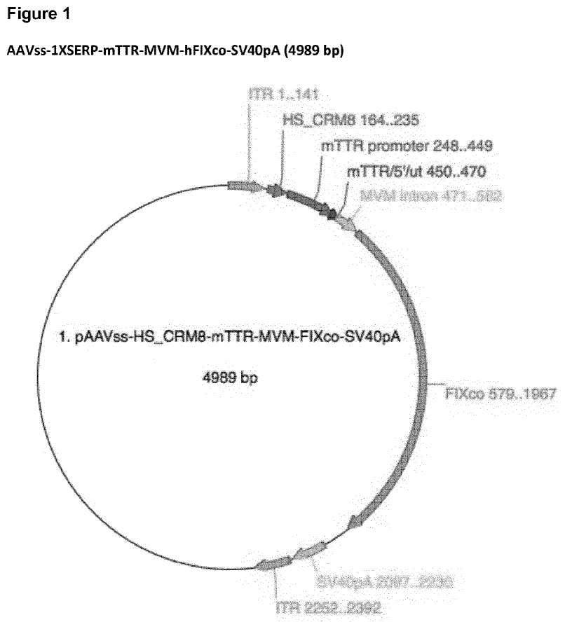

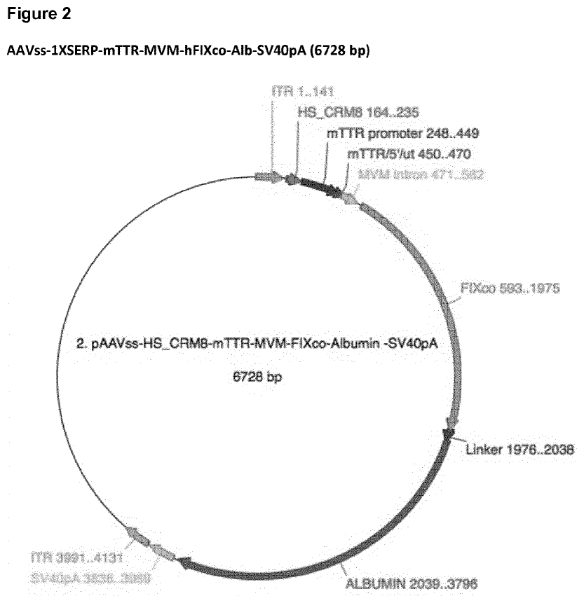

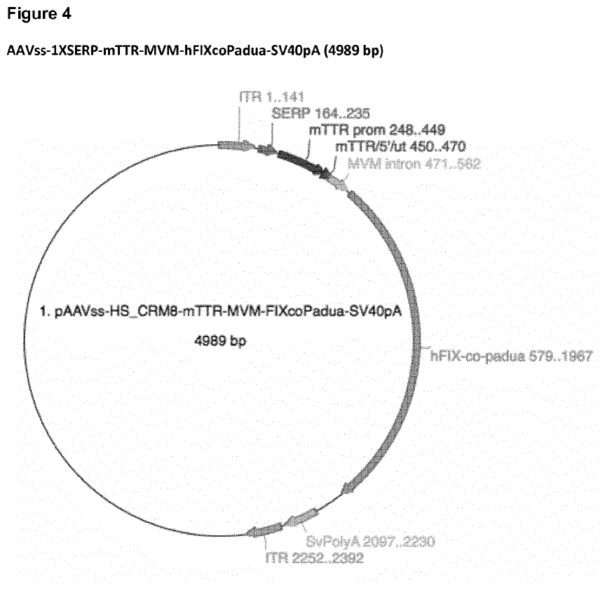

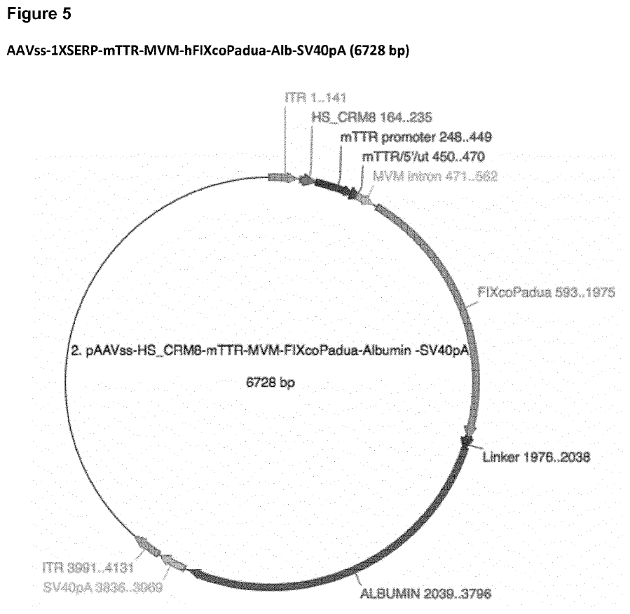

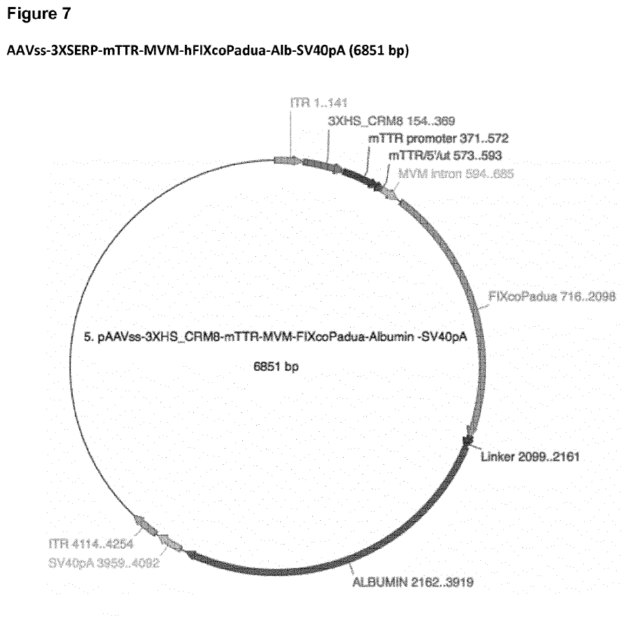

The corresponding vector plasmid maps and sequences are shown in FIGS. 1-8 and 12. AAVss corresponds to a single-stranded (ss) AAV vector backbone, as described previously in VandenDriessche et al., 2007. SERP correspond to a cis-regulatory element derived from the SERPINA1 gene, identical to HS-CRM8 in previous publications (Nair et al., 2014; Chuah et al., 2014). The vectors contain either a single copy of this SERP element (designated as 1XSERP) or a triplet repeat (designated as 3XSERP), as indicated. mTTR corresponds to a minimal transtherythin promoter and MVM corresponds to the minute virus of mice intron. The vectors also contain a 5′ untranslated region (5′ UTR) of the TTR gene, downstream of the TTR minimal promoter (mTTR). hFIXco corresponds to a codon-optimized human (h)FIX gene as described previously (Nair et al., 2014; Chuah et al., 2014). Padua refers to the R338L gain-of-function FIX mutation, originally described by Simioni and colleagues in thrombophilia patients (Simioni et al., 2009). Alb refers to the wild-type, non codon-optimized albumin sequence whereas Albco refers to the corresponding codon-optimized albumin sequence. SV40 pA corresponds to the SV40 polyadenylation site. The hFIXcoAlbco, hFIXcoPadua-Alb and hFIXcoPadua-Albco fusion construct contain a linker (SEQ ID NO: 18) allowing synthesis of a fusion protein, as described previously (Metzner et al., 2009; Santagostino et al., 2016). Generation and initial characterization of the codon-optimized FIX (coFIX) with the hyperactivating Padua mutation (i.e. FIX-Padua) and the hepatocyte-specific promoter were described previously (Cantore et al., 2012; Nair et al., 2014).

The sequences of the different FIX genes are as follows: hFIXco (SEQ ID NO: 9); hFIXco-Albco (SEQ ID NO: 10); hFIXcoPadua (SEQ ID NO: 11); hFIXcoPadua-Alb (SEQ ID NO: 12); hFIXcoPadua-Albco (SEQ ID NO: 13).

The AAV vectors were produced by co-transfecting 293T cells with plasmids containing the AAV vector and helper constructs encoding the AAV8-DJ capsid (Grimm et al., 2008; Gao et al., 2004), as described (Nair et al., 2014; Chuah et al., 2014). Vectors were purified by cesium chloride ultra-centrifugation and the vector titers were determined by quantitative real-time PCR with vector-specific primers, as described (Nair et al., 2014; Chuah et al., 2014). Adult C57Bl6 and hemophilia B mice (Wang et al., 1997) were injected intravenously at the indicated AAV vector doses. FIX antigen levels were determined by enzyme-linked immunosorbent assay (ELISA) and FIX activity was determined with a chromogenic assay (HYPHEN BioMed, Andresy, France) as described (Nair et al., 2014; Chuah et al., 2014) and per manufacturer's instructions Animal experiments were approved by the university's animal ethics committee. mRNA expression levels were determined by quantitative real-time PCR and quantitative real-time reverse transcriptase PCR, respectively, as described (Nair et al., 2014; Chuah et al., 2014).

2. Results

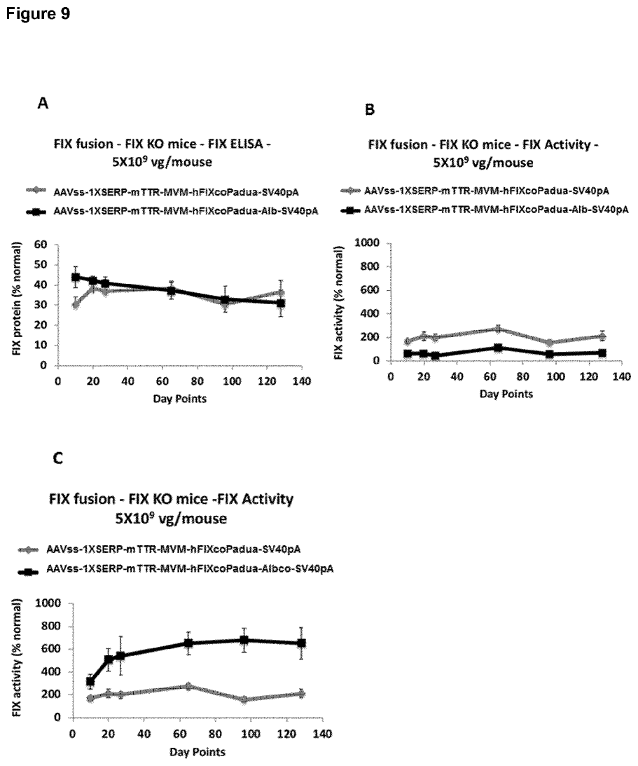

The applicants first assessed the impact of the codon-optimized versus non codon-optimized albumin fusion on the circulating human FIX levels and activity. Hemophilic FIX-deficient mice (FIX knock-out or FIX KO) were injected i.v. with 5×10⁹ vg/mouse of the AAVss-1XSERP-mTTR-MVM-hFIXcoPadua-SV40pA, AAVss-1XSERP-mTTR-MVM-hFIXcoPadua-Alb-SV40pA; AAVss-1XSERP-mTTR-MVM-hFIXcoPadua-Albco-SV40pA vectors. All vectors were packaged with the AAV8-DJ capsid. The results show that the AAV vectors encoding the codon-optimized hFIXcoPadua-Albco fusion protein (i.e. AAVss-1XSERP-mTTR-MVM-hFIXcoPadua-Albco-SV40pA) resulted in significantly higher FIX activity (about 3-fold), compared to the AAV vectors encoding the hFIXcoPadua protein, that was not fused to albumin (i.e. AAVss-1XSERP-mTTR-MVM-hFIXcoPadua-SV40pA) (FIG. 9A). The FIX activity levels were sustained in the treated animals, consistent with the lack of an anti-hFIX or anti-hAlb immune response.

In contrast, there was no significant difference in circulating FIX antigen levels in hemophilic FIX-deficient mice injected i.v. with 5×10⁹ vg/mouse of the AAVss-1XSERP-mTTR-MVM-hFIXcoPadua-SV40pA versus AAVss-1XSERP-mTTR-MVM-hFIXcoPadua-Alb-SV40pA vectors (FIG. 9B). Similarly, in contrast to when the hFIXcoPadua-Albco fusion gene was employed, circulating FIX activity levels in hemophilic FIX-deficient mice injected i.v. with 5×10⁹ vg/mouse of the AAVss-1XSERP-mTTR-MVM-hFIXcoPadua-Alb-SV40pA vector was similar or even slightly lower than the levels obtained with the AAVss-1XSERP-mTTR-MVM-hFIXcoPadua-SV40pA vector (FIG. 9C). Collectively, these results indicate that FIX-albumin fusions result in higher FIX activity levels provided that the appropriate codon-optimization is performed on the albumin fusion itself.

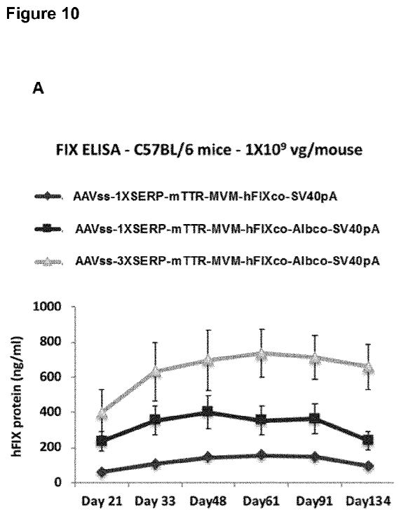

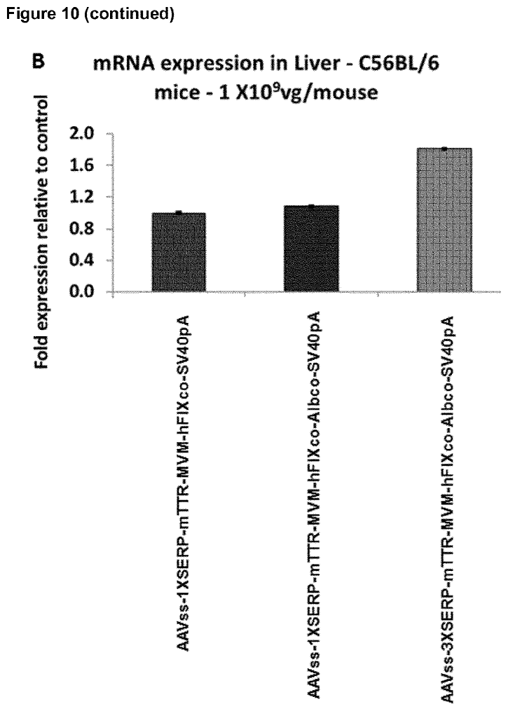

To confirm the effect of the albumin fusion, its impact on the FIX levels in wild-type C57Bl6 mice was subsequently assessed using the non-Padua hFIXco as therapeutic transgene. Normal C57Bl6 mice were therefore injected with 10⁹ vg/mouse of the AAVss-1XSERP-mTTR-MVM-hFIXco-SV40pA or AAVss-1XSERP-mTTR-MVM-hFIXco-Albco-SV40pA vectors. The results indicate that the albumin fusion significantly increases the circulating FIX antigen levels following injection of the AAVss-1XSERP-mTTR-MVM-hFIXco-Albco-SV40pA vector, compared to the non-fusion control (i.e. AAVss-1XSERP-mTTR-MVM-hFIXco-SV40pA) (FIG. 10A). Hence, the increased efficacy of gene therapy using FIX-albumin fusions can be obtained based on two different proteins i.e. hyperactive Padua FIX-R338L (FIG. 9) and wild-type FIX (FIG. 10) and is thus irrespective of the Padua FIX-R338L mutation.

Furthermore, FIG. 10B shows that the mRNA expression of FIX in liver between mice injected with AAVss-1XSERP-mTTR-MVM-hFIXco-SV40pA or AAVss-1XSERP-mTTR-MVM-hFIXco-Albco-SV40pA vectors is substantially the same, suggesting that the increased circulating FIX antigen levels following injection of the AAVss-1XSERP-mTTR-MVM-hFIXco-Albco-SV40pA vector compared to the non-fusion control (i.e. AAVss-1XSERP-mTTR-MVM-hFIXco-SV40pA) (FIG. 10A) can be attributed to an increased half-life of the hFIX-albumin fusion protein, rather than to an increased mRNA expression thereof.

To further increase the therapeutic effect of the FIX-albumin gene therapy approach, we assessed whether including multiple copies of the SERP element further increased the steady-state FIX levels. Normal C57Bl6 mice were therefore injected with 10⁹ vg/mouse of the AAVss-1XSERP-mTTR-MVM-hFIXco-Albco-SV40pA or AAVss-3XSERP-mTTR-MVM-hFIXco-Albco-SV40pA vectors. The results showed that multiple copies of the SERP element augmented the circulating levels of the hFIX-Albumin fusion protein (FIG. 10A), consistent with a significant increase in hFIXco-Albco mRNA levels (FIG. 10B).

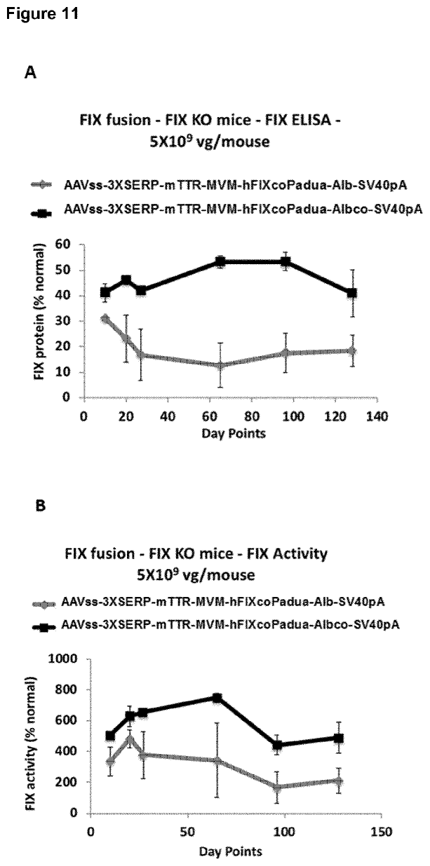

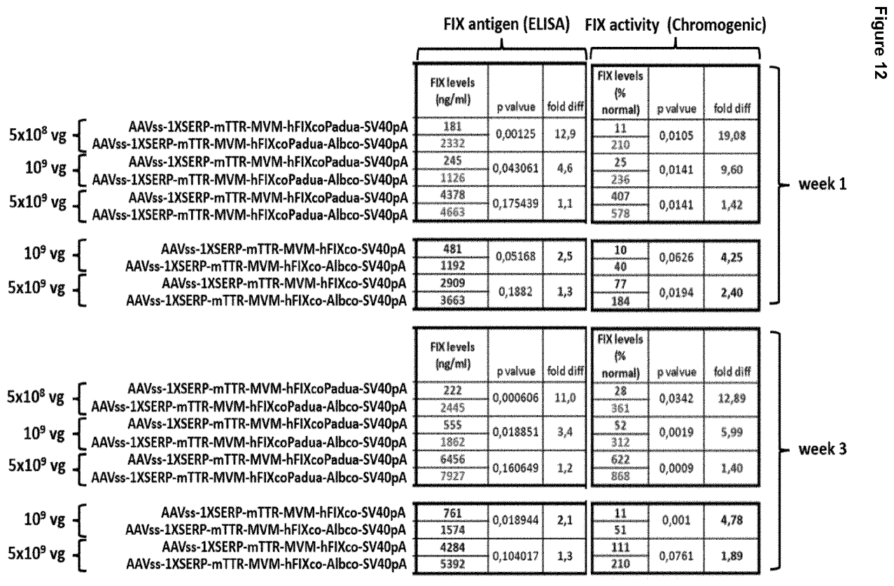

Finally, using the optimized vector design based on multiple SERP elements, we confirmed the superior therapeutic efficacy of the hFIXcoPadua-Albco transgene compared to hFIXcoPadua-Alb. Hemophilic FIX-deficient mice were therefore injected with 5×10⁹ vg/mouse of the AAVss-3XSERP-mTTR-MVM-hFIXcoPadua-Alb-SV40pA or AAVss-3XSERP-mTTR-MVM-hFIXcoPadua-Albco-SV40pA vectors. The results show that the AAV vectors encoding the codon-optimized hFIXcoPadua-Albco fusion protein (i.e. AAVss-3XSERP-mTTR-MVM-hFIXcoPadua-Albco-SV40pA) resulted in significantly higher FIX antigen and activity levels (about 2 to 4-fold), compared to the antigen and activity levels AAV vectors encoding the hFIXcoPadua protein, that was not fused to albumin (i e AAVss-3XSERP-mTTR-MVM-hFIXcoPadua-SV40pA) (FIGS. 11A and B).

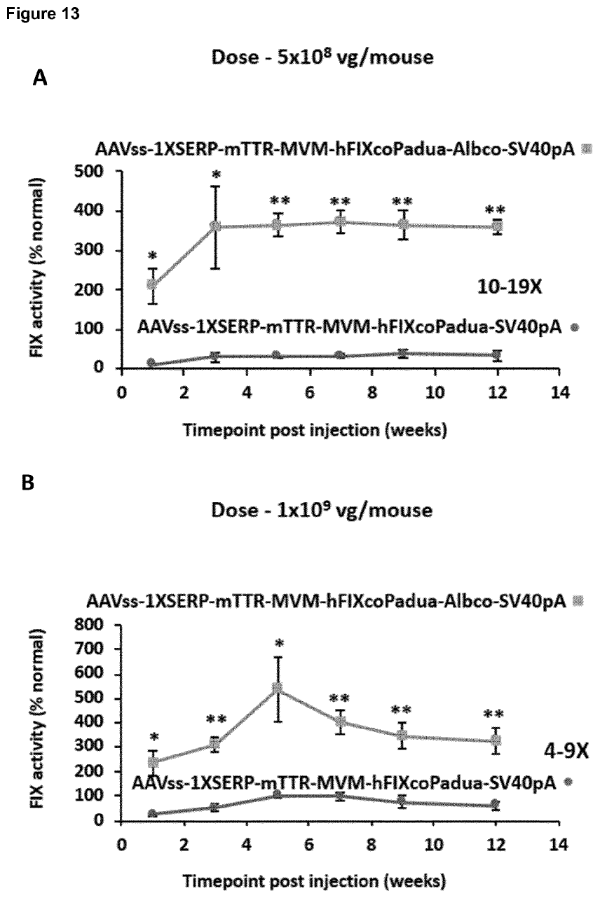

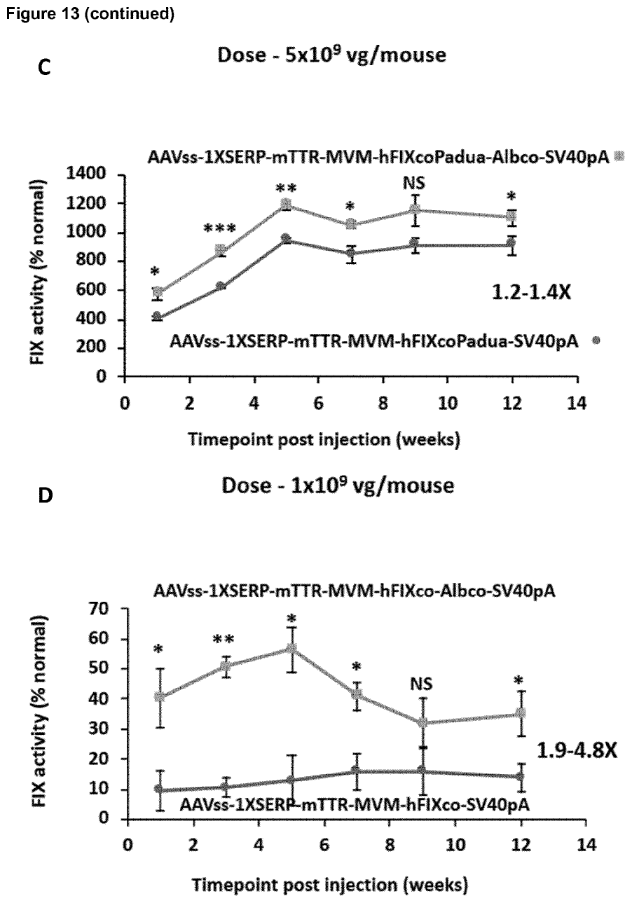

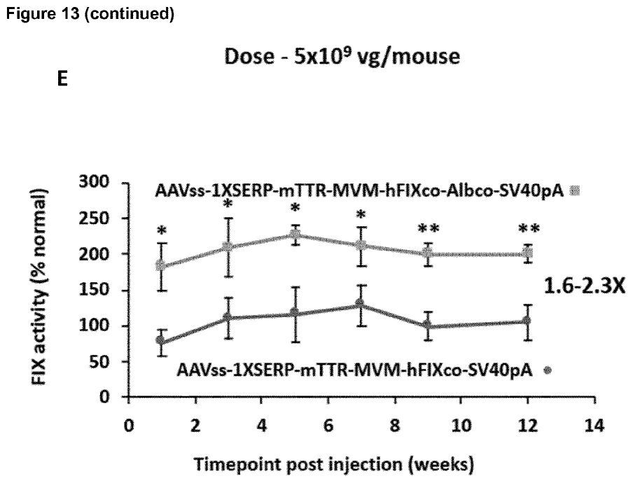

To further confirm the effect of the albumin fusion, its impact on the FIX antigen levels and FIX activity was subsequently assessed in hemophilic FIX following a comprehensive dose-response analysis, using either the non-Padua hFIXco or the Padua-hFIXco (“hFIXcoPadua”) as therapeutic transgenes. Hemophilic FIX-deficient mice were therefore injected with 5×10⁸ vg/mouse, 1×10⁹ vg/mouse or 5×10⁹ vg/mouse of the AAVss-1XSERP-mTTR-MVM-hFIXco-SV40pA (SEQ ID NO: 1), AAVss-1XSERP-mTTR-MVM-hFIXco-Albco-SV40pA (SEQ ID NO: 2), AAVss-1XSERP-mTTR-MVM-hFIXcoPadua-SV40pA (SEQ ID NO: 4) or AAVss-1XSERP-mTTR-MVM-hFIXcoPadua-Albco-SV40pA (SEQ ID NO: 6) vectors. FIX antigen and activity levels were measured 1 and 3 weeks after vector injection (FIGS. 12 and 13).

The results show that the AAV vectors encoding the codon-optimized hFIXcoPadua-Albco fusion protein (i.e. AAVss-1XSERP-mTTR-MVM-hFIXcoPadua-Albco-SV40pA) resulted in significantly higher FIX antigen and activity levels compared to the antigen and activity levels obtained with AAV vectors encoding the hFIXcoPadua protein, that was not fused to albumin or codon optimized albumin (“Albco”) (i.e. AAVss-1XSERP-mTTR-MVM-hFIXcoPadua-SV40pA) (FIGS. 12 and 13 A-C). Similarly, the results also indicated that the AAV vectors encoding the codon-optimized hFIXco-Albco fusion protein (i.e. AAVss-1XSERP-mTTR-MVM-hFIXco-Albco-SV40pA) resulted in significantly higher FIX antigen and activity levels compared to the antigen and activity levels obtained with AAV vectors encoding the hFIXco protein, that was not fused to albumin or codon optimized albumin (“Albco”) (i.e. AAVss-1XSERP-mTTR-MVM-hFIXco-SV40pA) (FIGS. 12 and 13 D-E). In addition, the FIX activity levels obtained with the AAV vectors encoding hFIXcoPadua were significantly higher than the activity levels obtained with the AAV vectors encoding the non-hyperactive hFIXco (FIGS. 12 and 13 A-E). This was consistently observed with either the albumin fusions or non-fusion controls. FIX antigen and activity levels increased with increasing vector doses (FIGS. 12 and 13 A-E). The highest FIX antigen and activity levels were attained after injected of 5×10⁹ vg/mouse of AAVss-1XSERP-mTTR-MVM-hFIXcoPadua-Albco-SV40pA (in the range of 1000-1200% FIX activity: 10 to 12-fold physiologic FIX levels) (FIGS. 12 and 13 C).

The difference in FIX antigen and activity levels between the AAVss-1XSERP-mTTR-MVM-hFIXcoPadua-Albco-SV40pA albumin fusion versus non-fusion AAVss-1XSERP-mTTR-MVM-hFIXcoPadua-SV40pA control construct was more pronounced at the lowest (5×10⁸ vg/mouse) (10 to 19-fold) (FIGS. 12 and 13 A) and mid vector doses (10⁹ vg/mouse) (4 to 9-fold) (FIGS. 12 and 13 B) than at the highest vector dose (5×10⁹ vg/mouse) (FIGS. 12 and 13 C), suggesting a possible saturation effect. Similarly, the difference in FIX antigen and activity levels between the AAVss-1XSERP-mTTR-MVM-hFIXco-Albco-SV40pA albumin fusion versus non-fusion AAVss-1XSERP-mTTR-MVM-hFIXco-SV40pA control construct was also more pronounced at the mid vector doses (10⁹ vg/mouse) (2 to 5-fold) (FIGS. 12 and 13 D) than at the highest vector dose (5×10⁹ vg/mouse) (FIGS. 12 and 13 E). Collectively, these results confirm that the increased efficacy of gene therapy using FIX-albumin fusions can be obtained based on two different proteins i.e. hyperactive Padua FIX-R338L and wild-type FIX and is thus irrespective of the Padua FIXR338L mutation.

### REFERENCES

ABSHIRE T, KENET G (2004). Recombinant factor VIIa: a review of efficacy, dosing regism and safety in patients with congenital and acquired factor VIII or IX inhibitors. J Thromb Haemost. 2(6):899-909.
ANDERSEN J T, DALHUS B, VIUFF D, ET AL. Extending serum half-life of albumin by engineering neonatal Fc receptor (FcRn) binding (2014). J Biol Chem; 289:13492-13502.
ANNONI A, BROWN B D, CANTORE A, SERGI L S, NALDINI L, and RONCAROLO M G. (2009). In vivo delivery of a microRNA-regulated transgene induces antigen-specific regulatory T cells and promotes immunologic tolerance. Blood 114, 5152-5161
ARRUDA V R, STEDMAN H H, HAURIGOT V, and BUCHLIS G. (2010). Peripheral transvenular delivery of adeno-associated viral vectors to skeletal muscle as a novel therapy for hemophilia B. Blood 115, 4678-88.
AXELROD J H, READ M S, BRINKHOUS K M, and VERMA I M. (1990). Phenotypic correction of factor IX deficiency in skin fibroblasts of hemophilic dogs. Proc Natl Acad Sci USA; 87, 5173-7.
BROWN B D, SHI C X, POWELL S HURLBUT D, GRAHAM F L, and LILLICRAP D. (2004). Helper-dependent adenoviral vectors mediate therapeutic factor VIII expression for several months with minimal accompanying toxicity in a canine model of severe hemophilia A. Blood 103, 804-10.
BROWN B D, CANTORE A, ANNONI A, SERGI L S, LOMBARDO A, DELLA VALLE P, D'ANGELO A, and NALDINI L. (2007). A microRNA-regulated lentiviral vector mediates stable correction of hemophilia B mice. Blood 110, 4144-52.
BRUNETTI-PIERRI N, GROVE N C, ZUO Y, EDWARDS R, PALMER D, CERULLO V, TERUYA J, N G P. Bioengineered factor IX molecules with increased catalytic activity improve the therapeutic index of gene therapy vectors for hemophilia B. Hum Gene Ther. 2009 May; 20(5):479-85.
BUCHLIS G, PODSAKOFF G M, RADU A, HAWK S M, FLAKE A W, MINGOZZI F, and HIGH K A. (2012). Factor IX expression in skeletal muscle of a severe hemophilia B patient 10 years after AAV-mediated gene transfer. Blood 119, 3038-41.
BUDKER V, ZHANG G, KNECHTLE S, WOLFF J A. Naked DNA delivered intraportally expresses efficiently in hepatocytes. (1996) Gene Ther. July; 3(7):593-8.
BUNTING S, ZHANG L, XIE L, BULLENS S, MAHIMKAR R, FONG S, SANDZA K, HARMON D, YATES B, HANDYSIDE B, SIHN C R, GALICIA N, TSURUDA L, O'NEILL C A, BAGRI A, COLOSI P, LONG S, VEHAR G, CARTER B. Gene Therapy with BMN 270 Results in Therapeutic Levels of FVIII in Mice and Primates and Normalization of Bleeding in Hemophilic Mice. Mol Ther. 2018 Feb. 7; 26(2):496-509.
CANTORE A, NAIR N, DELLA VALLE P, D I MATTEO M, MÀTRAI J, SANVITO F, BROMBIN C, D I SERIO C, D'ANGELO A, CHUAH M, NALDINI L, VANDENDRIESSCHE T. Hyper-functional coagulation factor IX improves the efficacy of gene therapy in hemophilic mice. Blood. 2012. Oct. 4.
CHANG, J., JIN, J., LOLLAR, P., BODE, W., BRANDSTETTER, H., HAMAGUCHI, N., STRAIGHT, D. L. &STAFFORD, D. W. (1998). Changing residue 338 in human factor IX from arginine to alanine causes an increase in catalytic activity. J Biol Chem 273(20): 12089-12094.
CHOWDHURY J R, GROSSMAN M, GUPTA S, CHOWDHURY N R, BAKER J R J R, WILSON J M. (1991) Long-term improvement of hypercholesterolemia after ex vivo gene therapy in LDLR-deficient rabbits. Science. December 20; 254(5039):1802-5.
CHUAH M K, SCHIEDNER G, THORREZ L, BROWN B, JOHNSTON M, GILLIJNS V, HERTEL S, VAN ROOIJEN N, LILLICRAP D, COLLEN D, VANDENDRIESSCHE T, and KOCHANEK S. (2003). Therapeutic factor VIII levels and negligible toxicity in mouse and dog models of hemophilia A following gene therapy with high-capacity adenoviral vectors. Blood 101, 1734-43.
CHUAH M K, NAIR N, VANDENDRIESSCHE T. Recent progress in gene therapy for hemophilia. Hum Gene Ther. 2012a June; 23(6):557-65.
CHUAH M K, NAIR N, VANDENDRIESSCHE T. Recent progress in gene therapy for hemophilia. Hum Gene Ther. 2012b June; 23(6):557-65.
CHUAH M K, VANDENDRIESSCHE T. Platelet-directed gene therapy overcomes inhibitory antibodies to factor VIII. J Thromb Haemost. 2012c August; 10(8):1566-9 CHUAH M K, PETRUS I, D E BLESER P, ET AL. Liver-specific transcriptional modules identified by genome-wide in silico analysis enable efficient gene therapy in mice and non-human primates. Mol Ther. 2014; 22(9):1605-1613.
DONSANTE A, MILLER D G, LI Y, VOGLER C, BRUNT E M, RUSSELL D W, and SANDS M S. (2007). AAV vector integration sites in mouse hepatocellular carcinoma. Science 317, 477.
DOBRZYNSKI E, FITZGERALD J C, CAO 0, MINGOZZI F, WANG L, and HERZOG R W (2006) Prevention of cytotoxic T lymphocyte responses to factor IX-expressing hepatocytes by gene transfer-induced regulatory T cells. Proc Natl Acad Sci USA 103, 4592-4597.
EHRHARDT A, and KAY M A. (2002). A new adenoviral helper-dependent vector results in long-term therapeutic levels of human coagulation factor IX at low doses in vivo. Blood 99, 3923-30.
FAUST S M, BELL P, CUTLER B J, ASHLEY S N, ZHU Y, RABINOWITZ J E, WILSON J M. CpG-depleted adeno-associated virus vectors evade immune detection. J Clin Invest. 2013 July; 123(7):2994-3001.
FIELDS P A, ARRUDA V R, ARMSTRONG E, KIRK CHU, MINGOZZI, F. HAGSTROM, J., HERZOG R, HIGH K A. (2001). Risk and prevention of anti-factor IX formation in AAV-mediated gene transfer in the context of a large deletion of F9. Mol. Ther. 4, 201-210.
FINN J D, NICHOLS T C, SVORONOS N, ET AL. The efficacy and the risk of immunogenicity of FIX Padua (R338L) in hemophilia B dogs treated by AAV muscle gene therapy. Blood. 2012 Nov. 29; 120(23):4521-3.
FOLLENZI A, BATTAGLIA M, LOMBARDO A, ANNONI A, RONCAROLO M G, and NALDINI L. (2004). Targeting lentiviral vector expression to hepatocytes limits transgene-specific immune response and establishes long-term expression of human antihemophilic factor IX in mice. Blood 103, 3700-9.
GAO G P, ALVIRA M R, WANG L, JOHNSTON J, WILSON J M. (2002). Novel adeno-associated viruses from rhesus monkeys as vectors for human gene therapy. Proc Natl Acad Sci USA 99, 11854-9.
GAO G, VANDENBERGH L H, ALVIRA M R LU Y, CALCEDO R, ZHOU X, and WILSON J M. (2004). Clades of Adeno-associated viruses are widely disseminated in human tissues. J. Viro 178, 6381-6388.
GEORGE L A, SULLIVAN S K, GIERMASZ A, ET AL. Hemophilia B Gene Therapy with a High-Specific-Activity Factor IX Variant. N Engl J Med. 2017; 377(23):2215-2227.
GOLOR G, BENSEN-KENNEDY D, HAFFNER S, EASTON R, JUNG K, MOISES T, LAWO J P, JOCH C, VELDMAN A (2013. J Thromb Haemost. 11(11):1977-85
GRIMM D, LEE J S, WANG L, ET AL. In vitro and in vivo gene therapy vector evolution via multispecies interbreeding and retargeting of adeno-associated viruses. J Virol. 2008; 82(12):5887-5911.
HERZOG R W, YANG E Y, COUTO L B, HAGSTROM J N, ELWELL D, FIELDS P A, BURTON M, BELLINGER D A, READ M S, BRINKHOUS K M, PODSAKOFF G M, NICHOLS T C, KURTZMAN G J, and HIGH K A. (1999). Long-term correction of canine hemophilia B by gene transfer of blood coagulation factor IX mediated by adeno-associated viral vector. Nat Med. 5, 56-63.
HERZOG R W, MOUNT J D, ARRUDA V R, HIGH K A, and LOTHROP C D Jr. (2001). Muscle-directed gene transfer and transient immune suppression result in sustained partial correction of canine hemophilia B caused by a null mutation. Mol Ther. 4, 192-200.
HERZOG R W, HAGSTROM J N, KUNG S H, TAI S J, WILSON J M, FISHER K J, and HIGH K A. (1997) Stable gene transfer and expression of human blood coagulation factor IX after intramuscular injection of recombinant adeno-associated virus. Proc Natl Acad Sci USA. 94, 5804-5809.
HERZOG R W, FIELDS P A, ARRUDA V R, BRUBAKER J O, ARMSTRONG E, MCCLINTOCK D, BELLINGER D A, COUTO L B, NICHOLS T C, HIGH K A (2002) Influence of vector dose on factor IX-specific T and B cell responses in muscle-directed gene therapy. Hum Gene Ther 13, 1281-1291.
HERZOG E, HARRIS S, M C EWEN A, HENSEN C, PRAGST I, DICKNEITE G, SCHULTE S, ZOLLNER S (2014) Thromb Res 134(2):495-502
HIGH K A. (2001). Gene Transfer as an approach to treating Hemophilia. Circ Res. 88, 137-144.
HIGH K A. (2011) Gene therapy for hemophilia: a long and winding road. J Thromb Haemost. 9 Suppl. 1: 2-11.
HORLING F, FALKER F, CHUAH M. (2017) Development of SHP648, Shire's high performing AAV8-based FIX gene therapy vector. Human Gene Therapy. 28(12):A18
BAINBRIDGE J, SMITH A J, BARKER S, et al. (2008) Effect of Gene Therapy on Visual Function in Leber's Congenital Amaurosis. N Engl J Med. 358, 2231-2239.
JIANG H, LILLICRAP D, and PATARROYO-WHITE S. (2006). Multiyear therapeutic benefit of AAV serotypes 2, 6, and 8 delivering factor VIII to hemophilia A mice and dogs. Blood. 108, 107-15.
KAO, C. Y., LIN, C. N., Y U, I. S., TAO, M. H., W U, H. L., SHI, G. Y., YANG, Y. L., KAO, J. T. &LIN, S. W. (2010). FIX-Triple, a gain-of-function factor IX variant, improves haemostasis in mouse models without increased risk of thrombosis. Thromb Haemost 104(2): 355-365.
KAY M A, BALEY P, ROTHENBERG S, LELAND F, FLEMING L, PONDER K P, LIU T, FINEGOLD M, DARLINGTON G, POKORNY W, WOO SLC. (1992) Expression of human alpha 1-antitrypsin in dogs after autologous transplantation of retroviral transduced hepatocytes. Proc Natl Acad Sci USA. January 1; 89(1):89-93.
KAY M A, MANNO C S, RAGNI M V, COUTO L B, MCCLELLAND A, GLADER B, CHEW A J, TAI S J, HERZOG R W, ARRUDA V, JOHNSON F, SCALLAN C, SKARSGARD E, FLAKE A W, and HIGH K A. (2000). Evidence for gene transfer and expression of factor IX in hemophilia B patients treated with an AAV vector. Nat Genet. 24, 257-61.
KISTNER A, GOSSEN M, ZIMMERMANN F, JERECIC J, ULLMER C, LYBBERT H, BUJARD H. (1996) Doxycycline-mediated quantitative and tissue-specific control of gene expression in transgenic mice. Proc Natl Acad Sci USA. October 1; 93(20): 10933-8.
KREN B T, UNGER G M, SJEKLOCHA L, TROSSEN A A, KORMAN V, DIETHELEM-OKITA B M, REDING M T, and STEER C J. (2009). Nanocapsule-delivered Sleeping Beauty mediates therapeutic Factor VIII expression in liver sinusoidal endothelial cells of hemophilia A mice. J Clin Invest. 19, 2086-99.
KURIYAMA S, YOSHIKAWA M, ISHIZAKA S, TSUJII T, LKENAKA K, KAGAWA T, MORITA N, MIKOSHIBA K. (1991) A potential approach for gene therapy targeting hepatoma using a liver-specific promoter on a retroviral vector. Cell Struct Funct. December; 16(6):503-10.
LI H, MALANI N, HAMILTON S R, SCHLACHTERMAN A, BUSSADORI G, EDMONSON S E, SHAH R, ARRUDA V R, MINGOZZI F, WRIGHT J F, BUSHMAN F D, and HIGH K A. (2011). Assessing the potential for AAV vector genotoxicity in a murine model. Blood. 117, 3311-9.
LIN, C. N., KAO, C. Y., MIAO, C. H., HAMAGUCHI, N., W U, H. L., SHI, G. Y., LIU, Y. L., HIGH, K. A. &LIN, S. W. (2010). Generation of a novel factor IX with augmented clotting activities in vitro and in vivo. J Thromb Haemost 8(8): 1773-1783.
LIU F, SONG Y, LIU D. (1999) Hydrodynamics-based transfection in animals by systemic administration of plasmid DNA. Gene Ther. July; 6(7):1258-66.
MANNO C S, PIERCE G F, and ARRUDA V R. (2006). Successful transduction of liver in hemophilia by AAV-Factor IX and limitations imposed by the host immune response. Nat Med. 12, 342-7.
MÁTÉS L, CHUAH M K, BELAY E, JERCHOW B, MANOJ N, ACOSTA-SANCHEZ A, GRZELA D P, SCHMITT A, BECKER K, MATRAI J, M A L, SAMARA-KUKO E, GYSEMANS C, PRYPUTNIEWICZ D, MISKEY C, FLETCHER B, VANDENDRIESSCHE T, IVICS Z, and IZSVAK Z. (2009). Molecular evolution of a novel hyperactive Sleeping Beauty transposase enables robust stable gene transfer in vertebrates. Nat Genet. 41, 753-61.
MARGARTITIS P, VALDER R A, ALJAMALI M, CAMIRE R M, SCHLACHTERMAN A, HIGH K A (2004) J Clin Invest. Novel therapeutic approach for hemophilia using gene delivery of an engineered secreted activated Factor VII. 113(7): 1025-1031.
MÁTRAI J, CHUAH M K, and VANDENDRIESSCHE T. (2010a). Pre clinical and clinical progress in hemophilia gene therapy. Curr Opin Hematol. 17, 387-92.
MÁTRAI J, CHUAH M K, and VANDENDRIESSCHE T. (2010b). Recent advances in lentiviral vector development and applications. Mol Ther. 18, 477-90.
MÁTRAI J, CANTORE A, BARTHOLOMAE C C, ANNONI A, WANG W, ACOSTA-SANCHEZ A, SAMARA-KUKO E, D E WAELE L, M A L, GENOVESE P, DAMO M, ARENS A, GOUDY K, NICHOLS T C, VON KALLE C, L CHUAH M K, RONCAROLO M G, SCHMIDT M, VANDENDRIESSCHE T, and NALDINI L. (2011). Hepatocyte-targeted expression by integrase-defective lentiviral vectors induces antigen-specific tolerance in mice with low genotoxic risk. Hepatology 53, 1696-707.
MATSUI H, SHIBATA M, BROWN B, LABELLE A, HEGADRON C, ANDREWS C, CHUAH M, VANDENDRIESSCHE T, MIAO C H, HOUGH C, and LILLICRAP D. (2009). A murine model for induction of long-term immunologic tolerance to factor VIII does not require persistent detectable levels of plasma factor VIII and involves contributions from Foxp3+T regulatory cells. Blood. 114, 677-85.
MATSUI H, HEGADORN C, OZELO M, BURNETT E, TUTTLE A, LABELLE A, McCARY P B Jr., NALDINI L, BROWN B, HOUGH C, and LILLICRAP D. (2011). A microRNA-regulated and GP64-pseudotyped lentiviral vector mediates stable expression of FVIII in a murine model of Hemophilia A. Mol Ther. 19, 723-30.
McCARTY D M, MONAHAN P E, and SAMULSKI R J. (2001). Self-complementary recombinant adeno-associated virus (scAAV) vectors promote efficient transduction independently of DNA synthesis. Gene Ther. 8, 1248-54.
McCARTY D M, F U H, MONAHAN P E, TOULSON C E, NAIK P, and SAMULSKI R J. (2003). Adeno-associated virus terminal repeat (TR) mutant generates self-complementary vectors to overcome the rate-limiting step to transduction in vivo. Gene Ther. 10, 2112-8.
MCINTOSH, J. ET AL. Therapeutic levels of FVIII following a single peripheral vein administration of rAAV vector encoding a novel human factor VIII variant. Blood (2013).
MENDELL J R, AL-ZAIDY S, SHELL R, ET AL. Single-Dose Gene-Replacement Therapy for Spinal Muscular Atrophy. N Engl J Med. 2017; 377(18):1713-1722.
METZNER H J, WEIMER T, KRONTHALER U, ET AL. Genetic fusion to albumin improves the pharmacokinetic properties of factor IX. Thromb Haemost. 2009; 102(4):634-644.
METZNER H J, PIPE S W, WEIMER T, SCHULTE S (2013) Extending the pharmacokinetic half-life of coagulation factors by fusion to recombinant albumin. Thromb Haemost. 110(5): 931-939
MIAO C H, OHASHI K, PATIJN G A, MEUSE L, Y E X, THOMPSON A R, and KAY M A. (2000). Inclusion of the hepatic locus control region, an intron, and untranslated region increases and stabilizes hepatic factor IX gene expression in vivo but not in vitro. Mol Ther. 1, 522-32.
MIAO H. Z., SIRACHAINAN N., PALMER L., et al. (2004). Bioengineering of coagulation factor VIII for improved secretion. Blood 103 (9):3412-3419.
MIESBACH W, MEIJER K, COPPENS M, ET AL. Gene therapy with adeno-associated virus vector S-human factor IX in adults with hemophilia B. Blood. 2018; 131:1022-1031.
MIESBACH W, MEIJER K, COPPENS M, KAMPMANN P, KLAMROTH R, SCHUTGENS R, TANGELDER M, CASTAMAN G, SCHWABLE J, BONIG H, SEIFRIED E, CATTANEO F, MEYER C, LEEBEEK F W G. Gene therapy with adeno-associated virus vector 5-human factor IX in adults with hemophilia B. Blood. 2018 Mar. 1; 131(9):1022-1031.
MILANOV, ET AL., 2012 Engineered factor IX variants bypass FVIII and correct hemophilia A phenotype in mice Blood 119:602-611.
MILLER A D. (1990) Retrovirus packaging cells. Hum Gene Ther. Spring; 1 (1):5-14.
MINGOZZI F, LIU Y L, DOBRZYNSKI E, KAUFHOLD A, LIU J H, WANG Y, ARRUDA V R, HIGH K A, and HERZOG R W. (2003). Induction of immune tolerance to coagulation factor IX antigen by in vivo hepatic gene transfer. J Clin Invest. 111, 1347-56.
MINGOZZI F, MAUS M V, HUI D J, SABATINO D E, MURPHY S L, RASKO J E, RAGINI M V, MANNO C S, SOMMER J, JIANG H, PIERCE G F, ERTL H C, and HIGH K A. (2007). CD8(+) T-cell responses to adeno-associated virus capsid in humans. Nat Med. 13, 419-22.
MONAHAN P E, SUN J, GUI T, ET AL. Employing a gain-of-function factor IX variant R338L to advance the efficacy and safety of hemophilia B human gene therapy: preclinical evaluation supporting an ongoing adeno-associated virus clinical trial. Hum Gene Ther. 2015 February; 26(2):69-81.
MONAHAN P, WALSH C, POWELL J. S. (2015) Update on a phase ½ open-label trial of BAX335, an adeno-associated virus 8 (AAV8) vector-based gene therapy program for hemophilia B. Journal of Thrombosis and Haemostasis. 13: 87
MOUNT J D, HERZOG R W, TILLSON D M, GOODMAN S A, ROBINSON N, MCCLELAND M L, BELLINGER D, NICHOLS T C, ARRUDA V R, LOTHROP C D J R, and HIGH K A. (2002). Sustained phenotypic correction of hemophilia B dogs with a factor IX null mutation by liver-directed gene therapy. Blood 99, 2670-6.
NAIR N, RINCON M Y, EVENS H, SARCAR S, DASTIDAR S, SAMARA-KUKO E, GHANDEHARIAN O, MAN VIECELLI H, THÖNY B, D E BLESER P, VANDENDRIESSCHE T, CHUAH M K. (2014). Computationally designed liver-specific transcriptional modules and hyperactive factor IX improve hepatic gene therapy. Blood 123, 3195-9.
NAIR N, RINCON M Y, EVENS H, ET AL. Computationally designed liver-specific transcriptional modules and hyperactive factor IX improve hepatic gene therapy. Blood. 2014; 123(20):3195-3199.
CRUDELE J M, FINN J D, SINER J I, ET AL. AAV liver expression of FIX-Padua prevents and eradicates FIX inhibitor without increasing thrombogenicity in hemophilia B dogs and mice. Blood. 2015 Mar. 5; 125(10):1553-61.
NALDINI L, BLOMER U, GALLAY P, ORY D, MULLIGAN R, GAGE F H, VERMA I M, TRONO D. (1996) In vivo gene delivery and stable transduction of nondividing cells by a lentiviral vector. Science. April 12; 272(5259):263-7.
NATHWANI A C, DAVIDOFF A M, HANAWA H, YUNYU H U, HOFFER F A, NIKANOROV A, SLAUGHTER C, N G CYC, ZHOU J, LOZIER J, MANDRELL T D, VANIN E F, and NIENHUIS A W. (2002). Sustained high-level expression of human factor IX (hFIX) after liver-targeted delivery of recombinant adeno-associated virus encoding the hFIX gene in rhesus macaques. Blood 100, 1662-1669.
NATHWANI A C, GRAY J T, N G C Y, ZHOU J, SPENCE Y, WADDINGTON S N, TUDDENHAM E G, KEMBALL COOK G, McINTOSH J, BOON-SPIJKER M, MERTENS K, DAVIDOFF A M. (2006). Self-complementary adeno-associated virus vectors containing a novel liver-specific human factor IX expression cassette enable highly efficient transduction of murine and nonhuman primate liver. Blood 107, 2653-61.
NATHWANI A C, TUDDENHAM E G, RANGARAJAN S, ROSALES C, MCINTOSH J, LINCH D C, CHOWDARY P, RIDDELL A, PIE A J, HARRINGTON C, O′BEIRNE J, SMITH K, PASI J, GLADER B, RUSTAGI P, NG C Y, KAY M A, ZHOU J, SPENCE Y, MORTON C L, ALLAY J, COLEMAN J, SLEEP S, CUNNINGHAM J M, SRIVASTAVA D, BASNER-TSCHAKARJAN E, MINGOZZI F, HIGH K A, GRAY J T, REISS U M, NIENHUIS A W, and DAVIDOFF A M. (2011). Adenovirus-associated virus vector-mediated gene transfer in hemophilia B. N Engl J Med. 365, 2357-2365.
NATHWANI A C, REISS U M, TUDDENHAM E G, ET AL. Long-term safety and efficacy of factor IX gene therapy in hemophilia B. N Engl J Med. 2014; 371(21):1994-2004.
OHLFEST J R, FRANDSEN J L, FRITZ S, LOBITZ P D, PERKINSON S G, CLARK K J, NELSESTUEN G, KEY N S, MCLVOR R S, HACKETT P B, and LARGAESPADA D A. (2004). Phenotypic correction and long-term expression of factor VIII in hemophilic mice by immunotolerization and nonviral gene transfer using the Sleeping Beauty transposon system. Blood 105, 2691-8.
NATHWANI A C, REISS U M, TUDDENHAM E G, ROSALES C, CHOWDARY P, MCINTOSH J, DELLA PERUTA M, LHERITEAU E, PATEL N, RAJ D, RIDDELL A, PIE J, RANGARAJAN S, BEVAN D, RECHT M, SHEN Y M, HALKA K G, BASNER-TSCHAKARJAN E, MINGOZZI F, HIGH K A, ALLAY J, KAY M A, NG C Y, ZHOU J, CANCIO M, MORTON C L, GRAY J T, SRIVASTAVA D, NIENHUIS A W, DAVIDOFF A M. Long-term safety and efficacy of factor IX gene therapy in hemophilia B. N Engl J Med. 2014 Nov. 20; 371(21):1994-2004.
NEGRIER C. (2016) Entering new areas in known fields: recombinant fusion protein linking recombinant factor VIIa with recombinant albumin (rVIIa-FP)-advancing the journey. Thromb Res. 141(3):59-512.
PETERS R T, LOW S C, KAMPHAUS G D, DUMONT J A, ET AL. Prolonged activity of factor IX as a monomeric Fc fusion protein. Blood. 2010; 115(10):2057-2064.
PETRUS, I., CHUAH, M. & VANDENDRIESSCHE, T. Gene therapy strategies for hemophilia: benefits versus risks. J Gene Med 12, 797-809 (2010).
SANDBERG H, ALMSTEDT A, BRANDT J, et al. (2001). Structural and functional characteristics of the B domain-deleted recombinant factor VIII proteint, r-VIII SQ. Thromb Haemost. 85(1): 93-100.
POWELL J S, PASI K J, RAGNI M V, ET AL. Phase 3 study of recombinant factor IX Fc fusion protein in hemophilia B. N Engl J Med. 2013; 369(24):2313-2323.
RANGARAJAN S, WALSH L, LESTER W, ET AL. AAV5-factor VIII gene transfer in severe hemophilia A. N Engl J Med. 2017; 377:2519-2530.
SANTAGOSTINO E, MARTINOWITZ U, LISSITCHKOV T, ET AL. Long-acting recombinant coagulation factor IX albumin fusion protein (rIX-FP) in hemophilia B: results of a phase 3 trial. Blood. 2016; 127(14):1761-1769.
SCHULTE (2008) Use of albumin fusion technology to prolong the half-life of recombinant factor VIIa. Thromb Res. 122(4): S14-19.
SCHUETTRUMPF, J., HERZOG, R. W., SCHLACHTERMAN, A., KAUFHOLD, A., STAFFORD, D. W. &ARRUDA, V. R. (2005). Factor IX variants improve gene therapy efficacy for hemophilia B. Blood 105(6): 2316-2323.
SIMIONI, P., TORMENE, D., TOGNIN, G., GAVASSO, S., BULATO, C., IACOBELLI, N. P., FINN, J. D., SPIEZIA, L., RADU, C. &ARRUDA, V. R. (2009). X-linked thrombophilia with a mutant factor IX (factor IX Padua). N Engl J Med 361(17): 1671-1675.
SNYDER R O, MIAO C H, PATIJN G A, SPRATT S K, DANOS O, NAGY D, GOWN A M, WINTHER B, MEUSE L, COHEN L K, THOMPSON A R, and KAY M A. (1997). Persistent and therapeutic concentrations of human factor IX in mice after hepatic gene transfer of recombinant AAV vectors. Nat Genet. 16, 270-276.
SNYDER R O, MIAO C, MEUSE L, TUBB J, DONAHUE B A, HUI-FENG LIN, STAFFORD D W, PATEL S, THOMPSON A R, NICHOLS T, READ M S, BELLINGER D A, BRINKHOUS K M, and KAY M A. (1999). Correction of hemophilia B in canine and murine models using recombinant adeno-associated viral vectors. Nat Med. 5, 64-70.
STROHL W R (2015) Fusion proteins for half-life extension fo biologics as a strategy to make biobetters. Biodrugs. 29(4):215-239.
TRAPNELL B C. (1993) Adenoviral vectors for gene transfer. Adv. Drug Del. Rev. 12: 185-199.
VANDENBERGHE L H, WANG L, SOMANATHAN S, ZHI Y, FIGUEREDO J, CALCEDO R, SANMIGUEL J, DESAI R A, CHEN C S, JOHNSTON J, GRANT R L, GAO G, and WILSON J M. (2006). Heparin binding directs activation of T cells against adeno-associated virus serotype 2 capsid. Nat Med. 12, 967-71.
VANDENDRIESSCHE T, VANSLEMBROUCK V, GOOVAERTS I, ZWINNEN H, VANDERHAEGHEN M L, COLLEN D, and CHUAH M K. (1999). Long-term expression of human coagulation factor VIII and correction of hemophilia A after in vivo retroviral gene transfer in factor VIII-deficient mice. Proc Natl Acad Sci USA. 96, 10379-84.
VANDENDRIESSCHE T, THORREZ L, NALDINI L, FOLLENZI A, MOONS L, ZWI BERNEMAN, COLLEN D, and CHUAH M K. (2002). Lentiviral vectors containing the human immunodeficiency virus type-1 central polypurine tract can efficiently transduce nondividing hepatocytes and antigen-presenting cells in vivo. Blood 100, 813-22.
VANDENDRIESSCHE T, THORREZ L, ACOSTA-SANCHEZ A, PETRUS I, WANG L, M A L, D E WAELE L, IWASAKI Y, GILLIJNS V, WILSON J M, COLLEN D, and CHUAH M K. (2007). Efficacy and safety of adeno-associated viral vectors based on serotype 8 and 9 vs. lentiviral vectors for hemophilia B gene therapy. J Thromb Haemost. 5, 16-24.
VANDENDRIESSCHE T, IVICS Z, IZSVAK Z, and CHUAH M K. (2009). Emerging potential of transposons for gene therapy and generation of induced pluripotent stem cells. Blood 114, 1461-8.
VANDENDRIESSCHE T, and CHUAH M K. (2012). Clinical progress in gene therapy: sustained partial correction of the bleeding disorder in patients suffering from severe hemophilia B. Hum Gene Ther. 23, 4-6.
Wang L, Zoppè M, Hackeng T M, Griffin J H, Lee K F, Verma I M. A factor IX-deficient mouse model for hemophilia B gene therapy. Proc Natl Acad Sci USA. 1997; 94(21):11563-11566.
WANG L, TAKABE K, BIDLINGMAIER S M, ILL C R, and VERMA I M. (1999). Sustained correction of bleeding disorder in hemophilia B mice by gene therapy. Proc Natl Acad Sci USA 96, 3906-3910.
WANG L, NICHOLS T C, READ M S, BELLINGER D A, and VERMA I M. (2000). Sustained expression of therapeutic level of factor IX in hemophilia B dogs by AAV-mediated gene therapy in liver. Mol Ther. 1, 154-158.
WANG L, CAO O, SWALM B, DOBRZYNSKI E, MINGOZZI F, and HERZOG R W (2005) Major role of local immune responses in antibody formation to factor IX in AAV gene transfer. Gene Ther 12, 1453-464.
WARD N J, BUCKLEY S M, WADDINGTON S N, VANDENDRIESSCHE T, CHUAH M K, NATHWANI A C, McLNTOSH J, TUDDENHAM E G, KINNON C, THRASHER A J, and McVEY J H (2010) Codon optimization of human factor VIII cDNAs leads to high-level expression. Blood 117, 798-807.
WARD, N. J. ET AL. Codon optimization of human factor VIII cDNAs leads to high-level expression. Blood 117, 798-807 (2011).
WEILLER M, SCHUSTER M, COULIBALY S, ET AL. Biopotency and efficacy of SHP648, a next-generation fix gene therapy vector. Mol Ther. 2018; 26(suppl):Abstract 822.
W U Z, SUN J, ZHANG T, YIN C, YIN F, VAN DYKE T, SAMULSKI R J, and MONAHAN P E. (2008). Optimization of self-complementary AAV vectors for liver-directed expression results in sustained correction of hemophilia B at low vector dose. Mol Ther. 16, 280-9.
X U L, GAO C, and SANDS M S. (2003). Neonatal or hepatocyte growth factor-potentiated adult gene therapy with a retroviral vector results in therapeutic levels of canine factor IX for hemophilia B. Blood 101, 3924-3932.
X U L, NICHOLS T C, SARKAR R, Mc CORQUODALE S, BELLINGER D A, PONDER K P. (2005). Absence of a desmopressin response after therapeutic expression of factor VIII in hemophilia A dogs with liver-directed neonatal gene therapy. Proc Natl Acad Sci USA 102, 6080-6085.
YAMADA T, IWASAKI Y, TADA H, IWABUKI H, CHUAH M K, VANDENDRIESSCHE T, FUKUDA H, KONDO A, UEDA M, SENO M, TANIZAWA K, KURODA S. (2003) Nanoparticles for the delivery of genes and drugs to human hepatocytes. Nat Biotechnol. August; 21 (8):885-90.
YANT S R, MEUSE L, CHIU W, IVICS Z, IZSVAK Z, and KAY M A. (2000). Somatic integration and long-term transgene expression in normal and haemophilic mice using a DNA transposon system. Nat Genet. 25, 35-41.
Yusa et al. A hyperactive piggyBac transposase for mammalian applications. Proc Natl Acad Sci USA. 2011; 108(4):1531-6.
ZHANG G, BUDKER V, WOLFF J A. (1999) High levels of foreign gene expression in hepatocytes after tail vein injections of naked plasmid DNA. Hum Gene Ther. July 1; 10(10):1735-7.
ZHONG L, LI B, MAH C S, GOVINDASAMY L, AGBANDJE-MCKENNA, COOPER M, HERZOG R W, ZOLOTUKHIN I, WARRINGTON J R. K H, WEIGEL-VAN AKEN K, HOBBS J A, ZOLOTUKHIN S, MUZYCZKA N, and SRIVASTAVA A (2008). Next generation of adeno-associated virus 2 vectors: point mutations in tyrosines lead to high-efficiency transduction at lower doses. Proc Natl Acad Sci USA 105, 7827-32.
ZOLLNER S, SCHUERMANN D, RAQUET E, MUELLER-COHRS J, WEIMER T, PRAGST I, DICKNEITE G, SCHULTE S. (2014). Pharmacological characteristics of a novel, recombinant fusion protein linking coagulation factor VIIa with albumin (rVIIa-FP). J Thromb Haemost. 12(2):220-228

1. A codon-optimised nucleic acid molecule encoding human albumin comprising a sequence defined by SEQ ID NO: 14 or a sequence having at least 80% sequence identity to said sequence.

2. The nucleic acid molecule according to claim 1, comprising a transgene fused to said sequence defined by SEQ ID NO: 14 or said sequence having at least 80% sequence identity to said sequence, optionally wherein said transgene is located at the 5′ end of said sequence defined by SEQ ID NO: 14 or said sequence having at least 80% sequence identity to said sequence.

3. The nucleic acid molecule according to claim 2, wherein said transgene is separated from said sequence defined by SEQ ID NO: 14 or said sequence having at least 80% sequence identity to said sequence by a sequence encoding one or more polypeptide or peptide linkers.

4. A method of achieving at least one of increased expression, circulating levels, or activity of a protein or polypeptide encoded by a transgene comprising using the nucleic acid molecule according to claim 1 to express the protein or polypeptide in a cell.

5. A nucleic acid expression cassette comprising the nucleic acid molecule according to claim 2, operably linked to a promoter.

6. The nucleic acid expression cassette according to claim 5, comprising at least one of
at least one tissue-specific nucleic acid regulatory element operably linked to the promoter and the nucleic acid molecule;
a minute virus of mice (MVM) intron; and
a transcriptional termination signal.

7. The nucleic acid expression cassette according to claim 5, wherein said transgene encodes a secretable therapeutic protein or a secretable immunogenic protein.

8. The nucleic acid expression cassette according to claim 7, wherein said transgene encodes for
coagulation factor IX (FIX);
coagulation factor VIII (FVIII); or.
the light chain and the heavy chain of coagulation factor VII (FVII) or factor FVIIa (FVIIa), optionally wherein the light chain of FVII or FVIIa is coupled to the heavy chain of FVII or FVIIa by one or more cleavable polypeptide or peptide linkers.

9. The nucleic acid expression cassette according to claim 6, wherein the at least one tissue-specific nucleic acid regulatory element is at least one liver-specific nucleic acid regulatory element.

10. The nucleic acid expression cassette according to claim 9, wherein the at least one liver-specific nucleic acid regulatory element consists of the Serpin enhancer defined by SEQ ID NO: 25 or a sequence having at least 95% identity to said sequence.

11. The nucleic acid expression cassette according to claim 7, wherein the promoter is a liver-specific promoter.

12. A vector comprising the nucleic acid expression cassette according to claim 5.

13. The vector according to claim 12, having SEQ ID NO: 2, SEQ ID NO: 3, SEQ ID NO: 6 or SEQ ID NO: 8.

14. A pharmaceutical composition comprising the vector according to claim 12, and a pharmaceutically acceptable carrier.

15. (canceled)

16. A method of treating a liver-related disorder in a subject in need of such a treatment, comprising administering a therapeutically effective amount of the vector according to claim 12.

17. An in vitro or ex vivo method for expressing a transgene product in liver cells comprising:
introducing the nucleic acid expression cassette according to claim 5 into the liver cells;
expressing the transgene product in the liver cells.

18. A codon-optimised nucleic acid molecule encoding human albumin comprising the sequence defined by SEQ ID NO: 14.

19. The method of claim 11, wherein said liver-specific promoter is selected from the group consisting of: the transthyretin (TTR) promoter, the minimal TTR promotor (TTRm), the AAT promoter, the albumin (ALB) promotor or minimal promoter, the apolipoprotein A1 (APOA1) promoter or minimal promoter, the complement factor B (CFB) promoter, the ketohexokinase (KHK) promoter, the hemopexin (HPX) promoter or minimal promoter, the nicotinamide Nmethyltransferase (NNMT) promoter or minimal promoter, the (liver) carboxylesterase 1 (CES1) promoter or minimal promoter, the protein C (PROC) promoter or minimal promoter, the apolipoprotein C3 (APOC3) promoter or minimal promoter, the mannan-binding lectin serine protease 2 (MASP2) promoter or minimal promoter, the hepcidin antimicrobial peptide (HAMP) promoter or minimal promoter, and the serpin peptidase inhibitor, clade C (antithrombin), member 1 (SERPINC1) promoter or minimal promoter.

20. The nucleic acid expression cassette according to claim 9, wherein the at least one liver-specific nucleic acid regulatory element consists of a triple repeat of the Serpin enhancer defined by SEQ ID NO: 25 or a sequence having at least 95% identity to said sequence.

21. The nucleic acid molecule according to claim 2, wherein said transgene is a codon optimized transgene.


SEQUENCE LISTING (35 sequences)

SEQ ID NO: 1 - DNA, 4989 bp, Artificial Sequence
  AAVss-1XSERP-mTTR-MVM-hFIXco-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagcccaccatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaatgaaagatggatttccaaggttaattcattggaattgaaaattaacagcccccccccccccccccctgcagatctcaagcttcgaattctgcagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 6728 bp, Artificial Sequence
  AAVss-1XSERP-mTTR-MVM-hFIXco-Albco-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 3 - DNA, 6851 bp, Artificial Sequence
  AAVss-3XSERP-mTTR-MVM-hFIXco-Albco-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtagggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 4 - DNA, 4989 bp, Artificial Sequence
  AAVss-1XSERP-mTTR-MVM-hFIXcoPadua-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagcccaccatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaatgaaagatggatttccaaggttaattcattggaattgaaaattaacagcccccccccccccccccctgcagatctcaagcttcgaattctgcagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 6728 bp, Artificial Sequence
  AAVss-1XSERP-mTTR-MVM-hFIXcoPadua-Alb-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataggtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 6728 bp, Artificial Sequence
  AAVss-1XSERP-mTTR-MVM-hFIXcoPadua-Albco-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 7 - DNA, 6851 bp, Artificial Sequence
  AAVss-3XSERP-mTTR-MVM-hFIXcoPadua-Alb-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtagggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataggtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 8 - DNA, 6851 bp, Artificial Sequence
  AAVss-3XSERP-mTTR-MVM-hFIXcoPadua-Albco-SV40pA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcggtagggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagggatggttggttggtggggtattaatgtttaattacctggagcacctgcctgaaatcactttttttcaggttggctagttctagaagatctcaagcttgctagcatgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaagtcgacaccggtagataactgatcggatctaggctcgacatgctttatttgtgaaatttgtgatgctattgctttatttgtaaccattataagctgcaataaacaagttaacaacaacaattgcattcattttatgtttcaggttcagggggaggtgtgggaggttttttaaactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 9 - DNA, 1389 bp, Artificial Sequence
  hFIXco
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaatga

SEQ ID NO: 10 - DNA, 3204 bp, Artificial Sequence
  hFIXco-Albco
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaa

SEQ ID NO: 11 - DNA, 1389 bp, Artificial Sequence
  hFIXcoPadua
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaatga

SEQ ID NO: 12 - DNA, 3204 bp, Artificial Sequence
  hFIXcoPadua-Alb
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatag

SEQ ID NO: 13 - DNA, 3204 bp, Artificial Sequence
  hFIXcoPadua-Albco
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggccgcgtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggccgcgacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctctgtgagccagacctccaagctcaccagggccgagactgtcttccctgatgtggacggatccgacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaa

SEQ ID NO: 14 - DNA, 1758 bp, Artificial Sequence
  Albco
gacgcccacaagagcgaggtggcccacagattcaaggacctgggcgaggaaaacttcaaggctctggtgctgatcgccttcgcccagtacctgcagcagtgtcccttcgaggaccacgtgaagctggtcaacgaagtgaccgagttcgccaagacctgcgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgataaccccaacctgccccggctcgtgcggcccgaggtggacgtgatgtgcaccgccttccacgacaacgaggaaaccttcctgaagaagtacctgtacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagagagcctttaaggcctgggctgtggcccggctgagccagagattccccaaggccgagtttgccgaggtgtccaagctggtcaccgacctcaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggacgtgtgcaagaactacgccgaggccaaggatgtgttcctgggcatgttcctgtatgagtacgcccgcagacaccccgactacagcgtggtgctgctgctgcggctggccaagacctacgagacaaccctggaaaagtgctgcgccgctgccgacccccacgagtgctacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacccccaccctggtggaagtgtcccggaacctgggcaaagtgggcagcaagtgctgcaagcaccctgaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttctccgccctggaagtggacgagacatacgtgcccaaagagttcaacgccgagacattcaccttccacgccgacatctgcaccctgagcgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaaaaagctggtggccgcctctcaggccgccctgggactctaa

SEQ ID NO: 15 - protein, 14 aa, Artificial Sequence
  Linker
SFSQNPPVLTRHQR

SEQ ID NO: 16 - DNA, 4377 bp, Artificial Sequence
  coFVIIIdeltaB
atgcagatcgagctgtccacctgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaatatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgggagaaagaagatgacaaggtgttccctggcggcagccacacctacgtgtggcaggtgctgaaagaaaacggccccatggcctccgaccccctgtgcctgacctacagctacctgagccacgtggacctggtgaaggacctgaacagcggcctgatcggcgctctgctcgtctgccgggagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtccggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcttgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactatatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagacattcaagacccgggaggccatccagcacgagagcggcatcctgggccccctgctgtacggcgaagtgggcgacacactgctgatcatcttcaagaaccaggctagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggcgtgcagctggaagatcccgagttccaggccagcaacatcatgcactccatcaatggctacgtgttcgactctctgcagctctccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagacagtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgaaccccggagcttcagccagaacccccccgtgctgacgcgtcaccagcgggagatcacccggacaaccctgcagtccgaccaggaagagatcgattacgacgacaccatcagcgtggagatgaagaaagaggatttcgatatctacgacgaggacgagaaccagagccccagaagcttccagaagaaaacccggcactacttcattgccgccgtggagaggctgtgggactacggcatgagttctagcccccacgtgctgcggaaccgggcccagagcggcagcgtgccccagttcaagaaagtggtgttccaggaattcacagacggcagcttcacccagcctctgtatagaggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgccgaaccccggaagaacttcgtgaagcccaacgaaaccaagacctacttctggaaagtgcagcaccacatggcccccaccaaggacgagttcgactgcaaggcctgggcctacttcagcgacgtggatctggaaaaggacgtgcactctggactgattggcccactcctggtctgccacactaacaccctcaaccccgcccacggccgccaggtgaccgtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagagaatccggtggtatctgctgtccatgggcagcaacgagaatatccacagcatccacttcagcggccacgtgttcaccgtgcggaagaaagaagagtacaagatggccctgtacaacctgtaccccggcgtgttcgagacagtggagatgctgcccagcaaggccggcatctggcgggtggagtgtctgatcggcgagcacctgcacgctggcatgagcaccctgtttctggtgtacagcaacaagtgccagaccccactgggcatggcctctggccacatccgggacttccagatcaccgcctccggccagtacggccagtgggcccccaagctggccagactgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaatgtggacagcagcggcatcaagcacaacatcttcaacccccccatcattgcccggtacatccggctgcaccccacccactacagcattagatccacactgagaatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtcaacaaccccaaagaatggctgcaggtggactttcagaaaaccatgaaggtgaccggcgtgaccacccagggcgtgaaaagcctgctgaccagcatgtacgtgaaagagtttctgatcagcagctctcaggatggccaccagtggaccctgttctttcagaacggcaaggtgaaagtgttccagggcaaccaggactccttcacccccgtggtgaactccctggacccccccctgctgacccgctacctgagaatccacccccagtcttgggtgcaccagatcgccctcaggatggaagtcctgggatgtgaggcccaggatctgtactgatga

SEQ ID NO: 17 - protein, 25 aa, Artificial Sequence
  linker
SSGGSGGSGGSGGSGGSGGSGGSGS

SEQ ID NO: 18 - protein, 21 aa, Artificial Sequence
  linker
SVSQTSKLTRAETVFPDVDGS

SEQ ID NO: 19 - DNA, 202 bp, Artificial Sequence
  TTRm (TTR minimal promotor)
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtc

SEQ ID NO: 20 - DNA, 92 bp, Artificial Sequence
  MVM intron
aagaggtaagggtttaagggatggttggttggtggggtattaatgtttaattacctggagcacctgcctgaaatcactttttttcaggttgg

SEQ ID NO: 21 - DNA, 49 bp, Artificial Sequence
  https//protect-us.mimecast.com/s/- aWvCgJD4lu37P9Ux5LPj?domain=synt.pa
aataaaagatctttattttcattagatctgtgtgttggttttttgtgtg

SEQ ID NO: 22 - DNA, 295 bp, Artificial Sequence
  BGHpolyA
gatctgagccgaattcctgcagcccgggggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggga

SEQ ID NO: 23 - DNA, 134 bp, Artificial Sequence
  SV40polyA
atgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 24 - DNA, 100 bp, Artificial Sequence
  TTRe
cactgggaggatgttgagtaagatggaaaactactgatgacccttgcagagacagagtattaggacatgtttgaacaggggccgggcgatcagcaggtag

SEQ ID NO: 25 - DNA, 72 bp, Artificial Sequence
  SerpEnh
gggggaggctgctggtgaatattaaccaaggtcaccccagttatcggaggagcaaacaggggctaagtccac

SEQ ID NO: 26 - DNA, 218 bp, Artificial Sequence
  3xSerp
gggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccaccgggggaggctgctggtgaatattaaccaaggtcaccccagttatcggaggagcaaacaggggctaagtccac

SEQ ID NO: 27 - protein, 585 aa, Homo sapiens [sequence omitted]

SEQ ID NO: 28 - DNA, 1758 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatag

SEQ ID NO: 29 - DNA, 63 bp, Artificial Sequence
  linker
tctgtgagccagacctccaagctcaccagggccgagactgtcttccctgatgtggacggatcc

SEQ ID NO: 30 - protein, 406 aa, Homo sapiens [sequence omitted]

SEQ ID NO: 31 - protein, 152 aa, Homo sapiens [sequence omitted]

SEQ ID NO: 32 - protein, 254 aa, Homo sapiens [sequence omitted]

SEQ ID NO: 33 - protein, 6 aa, Artificial Sequence
  linker
RKRRKR

SEQ ID NO: 34 - protein, 412 aa, Artificial Sequence
  hFVII-2RKR
ANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRRKRRKRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 35 - protein, 8 aa, Artificial Sequence
  linker
PRPSRKRR